(19) United States
(12) Patent Application Publication
(10) Pub. No.: US 2011/0207128 A1
(43) Pub. Date: Aug. 25, 2011

(54) METHODS AND KITS FOR DETERMINING BIOLOGICAL AGE AND LONGEVITY BASED ON GENE EXPRESSION PROFILES

(76) Inventors: Richard M. Cawthon, Salt Lake City, UT (US); Richard A. Kerber, Louisville, KY (US); Sandra J. Hasstedt, Salt Lake City, UT (US); Elizabeth O'Brien, Louisville, KY (US)

(21) Appl. No.: 13/028,910

(22) Filed: Feb. 16, 2011

Related U.S. Application Data
(60) Provisional application No. 61/304,958, filed on Feb. 16, 2010.

(51) Int. Cl. C12Q 1/68

(52) U.S. Cl. 435/6.11; 435/6.18; 435/6.1

(57) ABSTRACT

Described herein are methods of predicting the likelihood of survival in a subject. Additionally, described herein are methods of modulating survival in a subject.

### CROSS-REFERENCE TO RELATED PATENT APPLICATIONS

This application claims priority to U.S. Provisional Application No. 61/304,958 filed on Feb. 16, 2010, which is hereby incorporated herein by reference in its entirety.

### STATEMENT REGARDING FEDERALLY SPONSORED RESEARCH

This invention was made with government support under NIH grant R01-AG022095 and NIH grant R21-AG030034. The government has certain rights in the invention.

### FIELD OF THE INVENTION

The present invention relates generally to the fields of molecular biology and longevity research. More specifically, the invention concerns methods and compositions useful for predicting the likelihood of survival.

### BACKGROUND

Researchers have long attempted to formulate tests to predict overall health and or longevity in organisms including humans. Systematic genome-wide screens for engineered gene expression changes that increase lifespan have identified scores of “longevity genes,” by deletion in yeast, RNA interference in C. elegans and activation of expression in Drosophila. To date, two genome-wide studies of longevity in humans have been published. In one such study, Puca and colleagues performed a sib-pair linkage study of centenarians and near centenarians, and identified a region of interest on chromosome 4q25. Subsequent work by this group led to the conclusion that one or more variants of microsomal triglyceride transfer protein were responsible for this effect, although other groups have failed to confirm this association. More recently, the first genome-wide association study of longevity and correlates of longevity found numerous associations with nominal p-values of 0.001 or less, including some candidate genes such as FOX1α; however, none of those candidate genes clearly exceeded a threshold allowing for reliable, multiple hypothesis testing. Although “longevity genes” have been sought after for many years, very few have been discovered and even less have become of use. Therefore, what is needed are methods for predicting the likelihood of survival of a subject based on gene expression profiles. Moreover what is needed are methods of decreasing the risk of mortality in a subject through the modulation of gene expression profiles.

### BRIEF SUMMARY

In accordance with the purpose of this invention, as embodied and broadly described herein, this invention relates to methods of predicting the likelihood of survival in a subject. Additionally, as embodied and broadly described herein, this invention relates to methods of increasing the likelihood of survival in a subject. The methods generally involve determining the expression levels of one or more genes in the subject. Additionally, the methods generally involve modulating the expression levels of one or more genes in a subject.

Additional advantages of the described methods and compositions will be set forth in part in the description which follows, and in part will be understood from the description, or may be learned by practice of the described methods and compositions. The advantages of the described methods and compositionss will be realized and attained by means of the elements and combinations particularly pointed out in the claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the described methods and compositionss and together with the description, serve to explain the principles of the described methods and compositionss.

FIG. 1 shows the standardized linear effect of age on each of 2,151 expression levels among 104 CEU grandparents is plotted on the X axis against the standardized effect of each expression level on mortality (log hazard rate ratio). Larger red dots indicate the observed values; smaller black dots represent values generated over 100 of the 1000 random permutations of the phenotypic (age, sex, and survival) data associated with each expression vector. Positive values on the X axis indicate increased expression with increasing age; negative values indicate decreased expression with increasing age. Positive values on the Y axis indicate increased mortality risk with increased expression; negative values indicate decreased risk. The dashed line is drawn at the fiftieth largest χ² value observed in 1000 permutations of 2151 genes.

FIG. 2 shows the LASSO (least absolute shrinkage and selection operator) model of biological age. At each step, an additional gene may be added to or subtracted from the model. a) mean square classification error (MSE) estimated by cross-validation, for the observed data (black line), and 100 random permutations of phenotype data (blue lines); b) probability of observing MSE less than or equal to the observed MSE (blue), and p-value of biological age estimate as a predictor of mortality (red); dashed line is 0.05; c) slope estimates for gene expressions included in the 14-step model (solid bars), and the 28-step model (hashed bars), showing how the estimated effect of a gene changes as more genes are added to the model. All models between steps 14 and 28 are both significantly better at estimating biological age than models based on random data and significantly better at predicting future mortality than models based on age and sex alone.

FIG. 3 LASSO model of survival. At each step, an additional gene is added to or subtracted from the model. a) mean square classification error (MSE) estimated by cross-validation, for the observed data (black line), and 100 random permutations of phenotype data (blue lines); b) probability of observing MSE less than or equal to the observed MSE (blue), and p-value of overall model as a predictor of mortality (red); dashed line is 0.05; c) estimated interquartile relative risks for terms included at step 7, showing the estimated effect of a typical variation in gene expression on the relative risk of dying. Models with between 4 and 8 genes included are better at predicting future mortality than 90% of models generated from random data, with a minimum p-value of 0.06 at step 7.

FIG. 4 shows idealized representations of the shapes of the relationship between age at draw and expression levels observed in 2,151 always-expressed genes in three-generation CEU family data.

FIG. 5 shows the association with mortality of LASSO models that predict FEL on the basis of expression patterns alone. Models increase in inclusiveness from left to right. The cyan dots represent models from 1000 permutations of phenotype data. Models of FEL with 5-19 predictors were best at predicting futures survival.

FIG. 6 shows a trace of the nonzero coefficient estimates of the model after step 19. LRRFIP1, CDKN3, RNF13, F8, and AMD1 are also listed among the top 20 individual associations. They are the first five effects to enter the model, at which point the association with survival is strongest.

FIG. 7 shows the Z-scores for the association of each gene expression with FEL (x-axis) and mortality (y-axis). Black dots are the observed data; cyan dots represent random permutations of phenotypic (FEL and survival) data. The dotted polygon encloses 95% of the maximal permuted observations ranked by Fisher's X2. IQGAP1 is the only gene with combined significance <0.05 after adjusting for multiple comparisons. Note also the moderately strong negative correlation between the mortality and FEL effects (r=−0.36; p<10-15).

FIG. 8 shows that IQGAP1 stands out from other genes not only in the bivariate FEL vs mortality association, but also in broad-sense heritability (H2=0.87; p=2*10−7).

FIG. 9 shows the results of a preliminary GWAS of IQGAP1 expression in the CEU families (IQGAP1 is on 15q26.1). SNPs in this region are associated with a familial predisposition to longevity.

### DETAILED DESCRIPTION

The described methods and compositionss may be understood more readily by reference to the following detailed description of particular embodiments and the Example included therein and to the Figures and their previous and following description.

Described are materials, compositions, and components that can be used for, can be used in conjunction with, can be used in preparation for, or are products of the described methods and compositionss. These and other materials are described herein, and it is understood that when combinations, subsets, interactions, groups, etc. of these materials are described that while specific reference of each various individual and collective combinations and permutation of these compounds may not be explicitly described, each is specifically contemplated and described herein. For example, if a nucleic acid is described and discussed and a number of modifications that can be made to a number of molecules including the nucleic acid are discussed, each and every combination and permutation of nucleic acid and the modifications that are possible are specifically contemplated unless specifically indicated to the contrary. Thus, if a class of molecules A, B, and C are described as well as a class of molecules D, E, and F and an example of a combination molecule, A-D is described, then even if each is not individually recited, each is individually and collectively contemplated. Thus, in this example, each of the combinations A-E, A-F, B-D, B-E, B-F, C-D, C-E, and C-F are specifically contemplated and should be considered described from disclosure of A, B, and C; D, E, and F; and the example combination A-D. Likewise, any subset or combination of these is also specifically contemplated and described. Thus, for example, the sub-group of A-E, B-F, and C-E are specifically contemplated and should be considered described from disclosure of A, B, and C; D, E, and F; and the example combination A-D. This concept applies to all aspects of this application including, but not limited to, steps in methods of making and using the described compositions. Thus, if there are a variety of additional steps that can be performed it is understood that each of these additional steps can be performed with any specific embodiment or combination of embodiments of the described methods, and that each such combination is specifically contemplated and should be considered described.

Those skilled in the art will recognize, or be able to ascertain using no more than routine experimentation, many equivalents to the specific embodiments of the methods and compositionss described herein. Such equivalents are intended to be encompassed by the following claims.

It is understood that the described methods and compositionss are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. DEFINITIONS

Unless defined otherwise, all technical and scientific terms used herein have the same meanings as commonly understood by one of skill in the art to which the described methods and compositionss belong. Although any methods and materials similar or equivalent to those described herein can be used in the practice or testing of the present methods and compositionss, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a nucleic acid” includes a plurality of such nucleic acids, reference to “the nucleic acid” is a reference to one or more nucleic acids and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values described herein, and that each value is also herein described as “about” that particular value in addition to the value itself. For example, if the value “10” is described, then “about 10” is also described. It is also understood that when a value is described that “less than or equal to” the value, “greater than or equal to the value” and possible ranges between values are also described, as appropriately understood by the skilled artisan. For example, if the value “10” is described the “less than or equal to 10” as well as “greater than or equal to 10” is also described. It is also understood that the throughout the application, data is provided in a number of different formats, and that this data, represents endpoints and starting points, and ranges for any combination of the data points. For example, if a particular data point “10” and a particular data point 15 are described, it is understood that greater than, greater than or equal to, less than, less than or equal to, and equal to 10 and 15 are considered described as well as between 10 and 15. It is also understood that each unit between two particular units are also described. For example, if 10 and 15 are described, then 11, 12, 13, and 14 are also described. The word “or” as used herein means any one member of a particular list and also includes any combination of members of that list.

The term “pharmaceutically effective amount” (or interchangeably referred to herein as “an effective amount”) has its usual meaning in the art, i.e., an amount of a pharmaceutical that is capable of inducing an in vivo and/or clinical response that facilitates management, prophylaxis, or therapy. This term can encompass therapeutic or prophylactic effective amounts, or both. As used herein, the term “suitable” means fit for mammalian, preferably human, use and for the pharmaceutical purposes described herein.

The term “treatment” or “treating” means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms. In some embodiments, the term “treatment” or “treating” includes ameliorating the symptoms of, curing or healing, and preventing the development of a given disease.

The term “prophylaxis” is intended as an element of “treatment” to encompass both “preventing” and “suppressing,” as defined herein. It will be understood by those skilled in the art that in human medicine it is not always possible to distinguish between “preventing” and “suppressing” since the ultimate inductive event or events may be unknown, latent, or the patient is not ascertained until well after the occurrence of the event or events.

The term “subject” means an individual. In one aspect, the subject is a mammal such as a primate, and, more preferably, a human. Non-human primates include marmosets, monkeys, chimpanzees, gorillas, orangutans, and gibbons, to name a few. The term “subject” includes domesticated animals, such as cats, dogs, etc., livestock (for example, cattle (cows), horses, pigs, sheep, goats, etc.), laboratory animals (for example, ferret, chinchilla, mouse, rabbit, rat, gerbil, guinea pig, etc.) and avian species (for example, chickens, turkeys, ducks, pheasants, pigeons, doves, parrots, cockatoos, geese, etc.). Subjects can also include, but are not limited to fish (for example, zebrafish, goldfish, tilapia, salmon and trout), amphibians and reptiles.

As used herein, a “subject” is the same as a “patient,” and the terms can be used interchangeably.

As used herein, a “sample” or “biological sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

The terms “modulate” “modulated,” or “modulation” can mean either increasing or decreasing that which is being modulated. For example, “modulate” can mean either increasing or decreasing the likelihood of survival. “Modulate” can also mean either increasing or decreasing the expression levels of any of the genes or SNPs described herein. In the methods of the present invention, inhibiting transcription, or inhibiting translation of the genes can modulate the expression levels. Similarly, the activity of a gene product (for example, an mRNA, a polypeptide or a protein) can be inhibited, either directly or indirectly. Modulation in expression level does not have to be complete. For example, expression level can be modulated by about 10%, 20%, 30%, 40%, 50%, 60%, 70%, 80%, 90%, 95%, 99%, 100% or any percentage in between as compared to a control wherein the expression level has not been modulated.

As used herein, a “modulator” can mean a composition that can either increase or decrease the expression or activity of a gene or gene product such as a peptide. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition. For example, a “candidate modulator” can be an active agent or a therapeutic agent.

The term “active agent” or “therapeutic agent” is defined as an active agent, such as drug, chemotherapeutic agent, chemical compound, etc. For example, and not to be limiting an active agent or a therapeutic agent can be a naturally occurring molecule or may be a synthetic compound, including, for example and not to be limiting, a small molecule (e.g., a molecule having a molecular weight <1000), a peptide, a protein, an antibody, or a nucleic acid, such as an siRNA or an antisense molecule. An active or therapeutic agent can be used individually or in combination with any other active or therapeutic agent.

By “prevent” is meant to minimize the appearance or development of or to inhibit the occurrence of an event. For example, “prevent” can mean to minimize the appearance or development of or to inhibit cancer tissue from forming in a cell line or in a subject. By “prevent” can also mean to inhibit or prevent the expression of p53 gene or peptide, a p53 pathway gene or peptide, a tumorigenic gene or peptide. Prevention does not have to be complete. For example, prevention can be 10%, 20%, 30%, 40%, 50%, 60%, 70%, 80%, 90%, 95%, 99%, 100% or any percentage in between as compared to a control

As used herein, the term “gene” refers to polynucleotide sequences which encode protein products and encompass RNA, mRNA, cDNA, single stranded DNA, double stranded DNA and fragments thereof. Genes can include introns and exons and non-coding sequences that indirectly modulate the function of other sequences. It is understood that the polynucleotide sequences of a gene can include complimentary sequences (e.g., cDNA).

The term “gene sequence(s)” refers to gene(s), full-length genes or any portion thereof. “Gene sequences” can include natural genes or synthetic genes, or genes created through manipulation.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

“Peptide” as used herein refers to any peptide, oligopeptide, polypeptide, gene product, expression product, or protein. A peptide is comprised of consecutive amino acids. The term “peptide” encompasses naturally occurring or synthetic molecules.

As used herein, the term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

In addition, as used herein, the term “peptide” refers to amino acids joined to each other by peptide bonds or modified peptide bonds, e.g., peptide isosteres, etc. and may contain modified amino acids other than the 20 gene-encoded amino acids. The peptides can be modified by either natural processes, such as post-translational processing, or by chemical modification techniques which are well known in the art. Modifications can occur anywhere in the peptide, including the peptide backbone, the amino acid side-chains and the amino or carboxyl termini. The same type of modification can be present in the same or varying degrees at several sites in a given polypeptide. Also, a given peptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins-Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

“Differential expression” or “different expression” as used herein refers to the change in expression levels of genes, and/or proteins encoded by said genes, in cells, tissues, organs or systems upon exposure to an agent. As used herein, differential gene expression includes differential transcription and translation, as well as message stabilization. Differential gene expression encompasses both up- and down-regulation of gene expression.

“Naturally occurring” refers to an endogenous chemical moiety, such as a carbohydrate, polynucleotide or polypeptide sequence, i.e., one found in nature. Processing of naturally occurring moieties can occur in one or more steps, and these terms encompass all stages of processing including, but not limited to the metabolism of a non-active compound to an active compound. Conversely, a “non-naturally occurring” moiety refers to all other moieties, e.g., ones which do not occur in nature, such as recombinant polynucleotide sequences and non-naturally occurring carbohydrates.

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific a nucleic acid (for example, genes and/or mRNAs) have at least 80%-90% sequence complementarity, preferably at least 91%-95% sequence complementarity, more preferably at least 96%-99% sequence complementarity, and most preferably 100% sequence complementarity to the DNA binding domain of the p53 nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By “specifically hybridizes” is meant that a probe, primer, or oligonucleotide recognizes and physically interacts (that is, base-pairs) with a substantially complementary nucleic acid under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1× Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

Throughout the description and claims of this specification, the word “comprise” and variations of the word, such as “comprising” and “comprises,” means “including but not limited to,” and is not intended to exclude, for example, other additives, components, integers or steps.

Throughout this application, various publications are referenced. The disclosures of these publications in their entireties are hereby incorporated by reference into this application in order to more fully describe the state of the art to which this pertains. The references described are also individually and specifically incorporated by reference herein for the material contained in them that is discussed in the sentence in which the reference is relied upon.

### B. METHODS FOR PREDICTING THE LIKELIHOOD OF SURVIVAL

Described herein are methods relating to the prediction of the likelihood of survival. Several genes associated with an increase or decrease in mortality have been identified including, but not limited to, CDC42, CORO1A, AURKB, CBX5, IQGAP1, TERF2, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, PRIVI1, MKNK2, SH3BGRL, RNH1, TMEM142C, CDC6, USP1, EDF1, QDPR, PRKAR1A, EIF3S10, SF3B1, SAFB, RNF11, IFNA1, IFNA2, IFNA4, IFNA6, IFNA7, IFNA10, IFNA13, IFNA14, IFNA16, IFNA17, BCL10, MARCH7, BAT2, PSMD4, PSMD4P2, SEC24B, TANK, SFRS2IP, SMNDC1, MARCKS, AGL, HNRPH1, C1D, VPS4B, TERF2IP, KIF2C, ACTR2, SPAG5, MTF2, and EMP3. Expression level of any or all of these genes can be used to predict the likelihood of survival in a subject.

As used herein, the term “survival” generally describes the state of surviving or remaining alive. More specifically, a subject having an increased likelihood of survival can mean a subject having reduced, or decreased mortality or a decrease in the risk of mortality. Conversely, a subject having a decreased likelihood of survival can mean a subject having increased, or greater mortality or an increase in the risk of mortality.

1. Predicting Survival

Described herein are methods for predicting the likelihood of survival of a subject comprising: a) obtaining a sample from a subject at a first time point; b) obtaining a second sample from the same subject at a second time point; c) determining the level of expression of one or more genes for each of the time points, wherein the one or more genes is CDC42, CORO1A, AURKB, CBX5, IQGAP1, TERF2, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1; d) predicting the likelihood of survival of the subject by comparing the expression level of one or more of the genes at the first time point to the expression level of one or more of the genes at the second time point, wherein a change in the expression level of one or more of the genes is predictive of survival. In one aspect an increase in the expression of CDC42 or TERF2 indicates a decreased likelihood of survival. In another aspect an increase in the expression of CORO1A, AURKB, CBX5, IQGAP1, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1 indicates an increased likelihood of survival. As used herein, a sample can be, but is not limited to, a urine sample, a blood sample, a tissue sample, a saliva sample, an amniotic fluid sample, a cerebrospinal fluid sample, a tear sample, or any combination thereof. Furthermore, the difference between the first time point and the second time point can vary. For example, and not to be limiting, the difference between the first time point and the second time point can be less than an hour, 1 hour, 12 hours, 24 hours, 2 days, 15 days, 1 month, 3 months, 6 months, 9 months, 1 year, 2 years, 5 years, 10 years, 20 years, 50 years, greater than 50 years, or any other time points in between.

In another aspect, a decrease in the expression of CDC42 or TERF2 indicates an increased likelihood of survival. In yet another aspect, a decrease in the expression of CORO1A, AURKB, CBX5, IQGAP1, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1 indicates a decreased likelihood of survival.

As used herein, “a change in expression level” can mean either an increase or a decrease in expression level. Increased or decreased expression does not have to be complete as this can range from a slight increase or decrease in expression to complete increase or decrease of expression. For example, expression can be increased or decreased by about 10%, 20%, 30%, 40%, 50%, 60%, 70%, 80%, 90%, 95%, 99%, 100% or any percentage in between.

The level of expression can be determined by any method known in the art for determining the expression levels of genes and gene products including, but not limited to, antibody or aptamer binding to a protein encoded by a gene, hybridization of mRNA or cDNA to a microarray, sequence specific probe hybridization, sequence specific amplification and similar methods.

The methods described herein can further comprise determining telomere length of the subject. For example, disclosed herein are methods that comprise determining telomere length of the subject, and correlating the telomere length with survival with telomere length in an age matched population of the subject. In one aspect the telomere length is the average telomere length. The telomere length can be determined by polymerase chain reaction performed on a sample from the subject. In one aspect, the sample can be, but is not limited to, blood or lymphoid cells. More specifically, in one aspect, the lymphoid cells can comprise T-cells.

As used herein, “age matched population” can mean within about 1, 2, 3, 4, 5, 6, 7, 8, 9, or 10 years of the age of the subject.

In one aspect, telomere length can be determined for a single chromosome in a cell. In another embodiment, the average telomere length or mean telomere length is measured for a single cell, and more preferably for a population of cells. A change in telomere length is an increase or decrease in telomere length, in particular an increase or decrease in the average telomere length. The change may be relative to a particular time point, i.e., telomere length of an organism at time point 1 as compared to telomere length at some later time point 2. A change or difference in telomere length may also be compared as against the average or mean telomere length of a particular cell population or organismal population, preferably those members of a population not suffering from a disease condition. In certain embodiments, change in telomere length is measured against a population existing at different time periods.

Although, telomere lengths can be determined for all eukaryotes, in a preferred embodiment, telomere lengths are determined for vertebrates, including without limitation, amphibians, birds, and mammals, for example rodents, ungulates, and primates, particularly humans. Preferred are organisms in which longevity is a desirable trait or where longevity and susceptibility to disease are correlated. In another aspect, the telomeres can be measured for cloned organisms in order to assess the mortality risk or disease susceptibility associated with altered telomere integrity in these organisms.

Samples for measuring telomeres are made using methods well known in the art. The telomere containing samples may be obtained from any tissue of any organism, including tissues of blood, brain, bone marrow, lymph, liver spleen, breast, and other tissues, including those obtained from biopsy samples. T issue and cells may be frozen or intact. The samples may also comprise bodily fluids, such as saliva, urine, feces, cerebrospinal fluid, semen, etc. Preferably, the tissue or cells are non-stem cells, i.e., somatic cells since the telomeres of stem cells generally do not decrease over time due to continued expression of telomerase activity. However, in some embodiments, telomeres may be measured for stems cells in order to assess inherited telomere characteristics of an organism.

Telomeric nucleic acids, or a target nucleic acid, may be any length, with the understanding that longer sequences are more specific. In some embodiments, it may be desirable to fragment or cleave the sample nucleic acid into fragments of 100-10,000 base pairs, with fragments of roughly 500 basepairs being preferred in some embodiments. Fragmentation or cleavage may be done in any number of ways well known to those skilled in the art, including mechanical, chemical, and enzymatic methods. Thus, the nucleic acids may be subjected to sonication, French press, shearing, or treated with nucleases (e.g., DNase, restriction enzymes, RNase etc.), or chemical cleavage agents (e.g., acid/piperidine, hydrazine/piperidine, iron-EDTA complexes, 1,10-phenanthroline-copper complexes, etc.).

The samples containing telomere and target nucleic acids can be prepared using techniques well-known in the art. For instance, the sample can be treated using detergents, sonication, electroporation, denaturants, etc., to disrupt the cells. The target nucleic acids can be purified as needed. Components of the reaction can be added simultaneously, or sequentially, in any order as outlined below. In addition, a variety of agents can be added to the reaction to facilitate optimal hybridization, amplification, and detection. These include salts, buffers, neutral proteins, detergents, etc. Other agents can be added to improve efficiency of the reaction, such as protease inhibitors, nuclease inhibitors, anti-microbial agents, etc., depending on the sample preparation methods and purity of the target nucleic acid. When the telomere nucleic acid is in the form of RNA, these nucleic acids may be converted to DNA, for example by treatment with reverse transcriptase (e.g., MoMuLV reverse transcriptase, Tth reverse transcriptase, etc.), as is well known in the art.

Numerous methods are available for determining telomere length. In one aspect, telomere length can be determined by measuring the mean length of a terminal restriction fragment (TRF). The TRF is defined as the length—in general the average length—of fragments resulting from complete digestion of genomic DNA with a restriction enzyme that does not cleave the nucleic acid within the telomeric sequence. Typically, the DNA is digested with restriction enzymes that cleaves frequently within genomic DNA but does not cleave within telomere sequences. Typically, the restriction enzymes have a four base recognition sequence (e.g., AluI, HinfI, RsaI, and Sau3A1) and are used either alone or in combination. The resulting terminal restriction fragment contains both telomeric repeats and subtelomeric DNA. As used herein, subtelomeric DNA are DNA sequences adjacent to tandem repeats of telomeric sequences and contain telomere repeat sequences interspersed with variable telomeric-like sequences. The digested DNA is separated by electrophoresis and blotted onto a support, such as a membrane. The fragments containing telomere sequences are detected by hybridizing a probe, i.e., labeled repeat sequences, to the membrane. Upon visualization of the telomere containing fragments, the mean lengths of terminal restriction fragments can be calculated (Harley, C. B. et al., Nature. 345(6274):458-60 (1990), hereby incorporated by reference). TRF estimation by Southern blotting gives a distribution of telomere length in the cells or tissue, and thus the mean telomere length of all cells.

For the various methods described herein, a variety of hybridization conditions may be used, including high, moderate, and low stringency conditions (see, e.g., Sambrook, J. Molecular Cloning: A Laboratory Manual, 3rd Ed., Cold Spring Harbor Laboratory Press, Cold Spring Harbor, N.Y. (2001); Ausubel, F. M. et al., Current Protocols in Molecular Biology, John Wiley & Sons (updates to 2002); hereby incorporated by reference). Stringency conditions are sequence-dependent and will be different in different circumstances, including the length of probe or primer, number of mismatches, G/C content, and ionic strength. A guide to hybridization of nucleic acids is provided in Tijssen, P. “Overview of Principles of Hybridization and the Strategy of Nucleic Acid Assays,” in Laboratory Techniques in Biochemistry and Molecular Biology: Hybridization with Nucleic Acid Probes, Vol 24, Elsevier Publishers, Amsterdam (1993). Generally, stringent conditions are selected to be about 5-10.degree. C. lower than the thermal melting point (i.e., Tₘ) for a specific hybrid at a defined temperature under a defined solution condition at which 50% of the probe or primer is hybridized to the target nucleic acid at equilibrium. Since the degree of stringency is generally determined by the difference in the hybridization temperature and the Tₘ, a particular degree of stringency may be maintained despite changes in solution condition of hybridization as long as the difference in temperature from Tₘ is maintained. The hybridization conditions may also vary with the type of nucleic acid backbone, for example ribonucleic acid or peptide nucleic acid backbone.

In another aspect, telomere length can be measured by quantitative fluorescent in situ hybridization (Q-FISH). In this method, cells are fixed and hybridized with a probe conjugated to a fluorescent label, for example, Cy-3, fluoresceine, rhodamine, etc. Probes for this method are oligonucleotides designed to hybridize specifically to telomere sequences. Generally, the probes are 8 or more nucleotides in length, preferably 12-20 more nucleotides in length. In one aspect, the probes are oligonucleotides comprising naturally occurring nucleotides. In one aspect, the probe is a peptide nucleic acid, which has a higher Tₘ than analogous natural sequences, and thus permits use of more stringent hybridization conditions. Generally, cells are treated with an agent, such as colcemid, to induce cell cycle arrest at metaphase provide metaphase chromosomes for hybridization and analysis. Digital images of intact metaphase chromosomes are acquired and the fluorescence intensity of probes hybridized to telomeres quantitated. This permits measurement of telomere length of individual chromosomes, in addition to average telomere length in a cell, and avoids problems associated with the presence of subtelomeric DNA (Zjilmans, J. M. et al., Proc. Natl. Acad Sci. USA 94:7423-7428 (1997); Blasco, M. A. et al., Cell 91:25-34 (1997); incorporated by reference).

In another aspect, telomere lengths can be measured by flow cytometry (Hultdin, M. et al., Nucleic Acids Res. 26: 3651-3656 (1998); Rufer, N. et al., Nat. Biotechnol. 16:743-747 (1998); incorporated herein by reference). Flow cytometry methods are variations of FISH techniques. If the starting material is tissue, a cell suspension is made, generally by mechanical separation and/or treatment with proteases. Cells are fixed with a fixative and hybridized with a telomere sequence specific probe, preferably a PNA probe, labeled with a fluorescent label. Following hybridization, cell are washed and then analyzed by FACS. Fluorescence signal is measured for cells in G.sub.O/G.sub.1 following appropriate subtraction for background fluorescence. This technique is suitable for rapid estimation of telomere length for large numbers of samples. Similar to TRF, telomere length is the average length of telomeres within the cell.

In another aspect, telomere lengths are determined by assessing the average telomere length using polymerase chain reaction (PCR). Procedures for PCR are widely used and well known (see for example, U.S. Pat. Nos. 4,683,195 and 4,683,202). In brief, a target nucleic acid is incubated in the presence of primers, which hybridizes to the target nucleic acid. When the target nucleic acid is double stranded, they are first denatured to generate a first single strand and a second single strand so as to allow hybridization of the primers. Any number of denaturation techniques may be used, such as temperature, although pH changes, denaturants, and other techniques may be applied as appropriate to the nature of the double stranded nucleic acid. A DNA polymerase is used to extend the hybridized primer, thus generating a new copy of the target nucleic acid. The synthesized duplex is denatured and the hybridization and extension steps repeated. Carrying out the amplification in the presence of a single primer results in amplification of the target nucleic acid in a linear manner. For the purposes of the present invention, linear amplification using a single primer is encompassed within the meaning of PCR. By reiterating the steps of denaturation, annealing, and extension in the presence of a second primer that hybridizes to the complementary target strand, the target nucleic acid encompassed by the two primers is amplified exponentially.

Also described herein are methods for predicting the likelihood of survival of a subject comprising determining the presence of one or more single nucleotide polymorphisms (SNPs) with an LOD score of greater than 3.5 with modulated expression of the IQGAP1 gene, wherein the presence of one or more single nucleotide polymorphisms (SNPs) with an LOD score of greater than 3.5 with modulated expression of the IQGAP1 gene is predictive of survival. The SNPs useful in the methods described herein include, but are not limited to rs716175, rs937793, rs3862432, rs3930162, rs17263706, rs3862434, rs8033595, rs12915189, rs7498042, rs12901137, rs12910489, rs12914286, rs7403002, rs11857476, rs7403440, rs10438448, or rs4344687. The term “IQGAP1” or “IQGAP1 gene” is meant to include genomic DNA encoding ras GTPase-activating-like protein (IQGAP1), including introns and exons, as well as 5′ and 3′ untranslated regions (UTR).

As used herein, “LOD score” means a statistical estimate of whether a SNP allele described herein is likely be associated with a change in IQGAP1 expression. LOD stands for logarithm of the odds to the base 10. An LOD score of three or more is generally taken to indicate that the SNP allele described herein is likely be associated with a change in IQGAP1 expression. A LOD score of three means the odds are a thousand to one in favor of genetic linkage.

Therefore, described herein are single nucleotide polymorphisms (SNPs) that can be used to predict the likelihood of survival in a subject and to select therapies for treating a subject at risk for decreased survival. A non-exhaustive list of SNPs for use in the described methods are provided in Table 1 below. Each SNP has at least two known alleles. Described is a consensus sequence for each SNP wherein the substituted residue can be identified with the actual residue in each individual allele (e.g. A, G, C, or T). However, also described is a sequence for each SNP where the identified residue is N (A, G, C, or T). Thus, in some aspects, the described methods comprise identifying a residue for each SNP location other than the one present in a control population. In other aspects, the method comprises identifying the residue for each SNP location identified as the 1^{st} or 2^{nd} allele.

TABLE 1
(SNPs) with an LOD score of greater than 3.5 with modulated expression of the IQGAP1 gene

| | LOD score with | | | | |
|---|---|---|---|---|---|
| | modulated | | | | |
| | expression of | Base | | | |
| SNP | the IQGAP1 gene | Change | Consensus | 1^{st} Allele | 2^{nd} Allele |
| rs716175 | 3.683 | C/T | SEQ ID NO: 1 | SEQ ID NO: 18 | SEQ ID NO: 19 |
| rs937793 | 3.713 | A/G | SEQ ID NO: 2 | SEQ ID NO: 20 | SEQ ID NO: 21 |
| rs3862432 | 4.047 | C/T | SEQ ID NO: 3 | SEQ ID NO: 22 | SEQ ID NO: 23 |
| rs3930162 | 3.986 | A/G | SEQ ID NO: 4 | SEQ ID NO: 24 | SEQ ID NO: 25 |
| rs17263706 | 3.897 | A/C | SEQ ID NO: 5 | SEQ ID NO: 26 | SEQ ID NO: 27 |
| rs3862434 | 3.986 | A/G | SEQ ID NO: 6 | SEQ ID NO: 28 | SEQ ID NO: 29 |
| rs8033595 | 3.807 | A/G | SEQ ID NO: 7 | SEQ ID NO: 30 | SEQ ID NO: 31 |
| rs12915189 | 3.929 | A/G | SEQ ID NO: 8 | SEQ ID NO: 32 | SEQ ID NO: 33 |
| rs7498042 | 3.851 | G/T | SEQ ID NO: 9 | SEQ ID NO: 34 | SEQ ID NO: 35 |
| rs12901137 | 4.267 | C/T | SEQ ID NO: 10 | SEQ ID NO: 36 | SEQ ID NO: 37 |
| rs12910489 | 4.411 | C/T | SEQ ID NO: 11 | SEQ ID NO: 38 | SEQ ID NO: 39 |
| rs12914286 | 4.267 | G/T | SEQ ID NO: 12 | SEQ ID NO: 40 | SEQ ID NO: 41 |
| rs7403002 | 4.106 | C/T | SEQ ID NO: 13 | SEQ ID NO: 42 | SEQ ID NO: 43 |
| rs11857476 | 3.761 | C/G | SEQ ID NO: 14 | SEQ ID NO: 44 | SEQ ID NO: 45 |
| rs7403440 | 4.251 | A/G | SEQ ID NO: 15 | SEQ ID NO: 46 | SEQ ID NO: 47 |
| rs10438448 | 4.564 | C/T | SEQ ID NO: 16 | SEQ ID NO: 48 | SEQ ID NO: 49 |
| rs4344687 | 4.808 | C/T | SEQ ID NO: 17 | SEQ ID NO: 50 | SEQ ID NO: 51 |

The methods described herein do not require detection of the substitution directly within the genomic DNA of the subject. The methods can comprise detecting nucleotides or amino acid residues in a sample that correspond to nucleotides with an LOD score of greater than 3.5 with modulated expression of the IQGAP1 gene within the subject. Thus, the methods can comprise detecting a nucleotide substitution in mRNA or cDNA that corresponds to a nucleotide with an LOD score of greater than 3.5 with modulated expression of the IQGAP1 gene within the subject.

The methods described herein can comprise identifying the residue corresponding to single nucleotide polymorphism (SNP) rs716175, rs937793, rs3862432, rs3930162, rs17263706, rs3862434, rs8033595, rs12915189, rs7498042, rs12901137, rs12910489, rs12914286, rs7403002, rs11857476, rs7403440, rs10438448, or rs4344687. For example, described herein are methods of predicting the likelihood of survival, comprising determining in a sample of nucleic acid from the subject the identity of one or more nucleotides with an LOD score of greater than 3.5 with modulated expression of the IQGAP1 gene, wherein a substitution of a nucleotide at one or more positions with an LOD score of greater than 3.5 with modulated expression of the IQGAP1 gene of the subject compared to a control indicates that the subject is at risk for decreased survival, wherein the method comprises identifying the residue corresponding to a single nucleotide polymorphism (SNP) at one or more of the following: rs716175, rs937793, rs3862432, rs3930162, rs17263706, rs3862434, rs8033595, rs12915189, rs7498042, rs12901137, rs12910489, rs12914286, rs7403002, rs11857476, rs7403440, rs10438448, or rs4344687.

The methods described herein can comprise identifying the residue corresponding to single nucleotide polymorphism (SNP) at a specific location, wherein a specific nucleic acid residue present is indicative of survival. For example described herein are methods that comprise identifying the residue corresponding to a single nucleotide polymorphism (SNP) at one or more of the following: rs716175, rs937793, rs3862432, rs3930162, rs17263706, rs3862434, rs8033595, rs12915189, rs7498042, rs12901137, rs12910489, rs12914286, rs7403002, rs11857476, rs7403440, rs10438448, or rs4344687, wherein: a cytosine (C) or thymine (T) nucleotide is at position 27 of the consensus sequence (SEQ ID NO:1) or a cytosine (C) or thymine (T) is at position 27 of SEQ ID NO:18 and SEQ ID NO:19 respectively; an adenine (A) or guanine (G) nucleotide is at position 27 of the consensus sequence (SEQ ID NO:2) or an adenine (A) or guanine (G) is at position 27 of SEQ ID NO:20 and SEQ ID NO:21, respectively; a cytosine (C) or thymine (T) nucleotide is at position 27 of the consensus sequence (SEQ ID NO:3), or a cytosine (C) or thymine (T) is at position 27 of SEQ ID NO:22 and SEQ ID NO:23, respectively; an adenine (A) or guanine (G) nucleotide is at position 27 of the consensus sequence (SEQ ID NO:4) or an adenine (A) or guanine (G) is at position 27 of SEQ ID NO:24 and SEQ ID NO:25, respectively; an adenine (A) or cytosine (C) nucleotide is at position 27 of the consensus sequence (SEQ ID NO:5) or an adenine (A) or cytosine (C) is at position 27 of SEQ ID NO:26 and SEQ ID NO:27, respectively; an adenine (A) or guanine (G) nucleotide is at position 27 of the consensus sequence (SEQ ID NO:6) or an adenine (A) or guanine (G) nucleotide is at position 27 of SEQ ID NO:28 and SEQ ID NO:29, respectively; an adenine (A) or guanine (G) nucleotide is at position 27 of the consensus sequence (SEQ ID NO:7) or an adenine (A) or guanine (G) at position 27 of SEQ ID NO:30 and SEQ ID NO:31, respectively; an adenine (A) or guanine (G) nucleotide is at position 27 of the consensus sequence (SEQ ID NO:8) or at position 27 of SEQ ID NO:32 and SEQ ID NO:33, respectively; a guanine (G) or thymine (T) nucleotide is at position 27 of the consensus sequence (SEQ ID NO:9) or at position 27 of SEQ ID NO:34 and SEQ ID NO:35 respectively, a cytosine (C) or thymine (T) nucleotide is at position 27 of the consensus sequence (SEQ ID NO:10) or a cytosine (C) or thymine (T) is at position 27 of SEQ ID NO:36 and SEQ ID NO:37, respectively; a cytosine (C) or thymine (T) nucleotide is at position 27 of the consensus sequence (SEQ ID NO:11) or a cytosine (C) or thymine (T) is at position 27 of SEQ ID NO:38 and SEQ ID NO:39, respectively; a guanine (G) or thymine (T) nucleotide is at position 27 of the consensus sequence (SEQ ID NO:12) or a guanine (G) or thymine (T) is at position 27 of SEQ ID NO:40 and SEQ ID NO:41, respectively; a cytosine (C) or thymine (T) nucleotide is at position 27 of the consensus sequence (SEQ ID NO:13) or a cytosine (C) or thymine (T) is at position 27 of SEQ ID NO:42 and SEQ ID NO:43, respectively; a cytosine (C) or guanine (G) nucleotide is at position 27 of the consensus sequence (SEQ ID NO:14) or a cytosine (C) or guanine (G) at position 27 of SEQ ID NO:44 and SEQ ID NO:45, respectively; an adenine (A) or guanine (G) nucleotide is at position 27 of the consensus sequence (SEQ ID NO:15) or an adenine (A) or guanine (G) is at position 27 of SEQ ID NO:46 and SEQ ID NO:47, respectively; a cytosine (C) or thymine (T) nucleotide is at position 27 of the consensus sequence (SEQ ID NO:16) or a cytosine (C) or thymine (T) is at position 27 of SEQ ID NO:48 and SEQ ID NO:49, respectively; or a cytosine (C) or thymine (T) nucleotide is at position 27 of the consensus sequence (SEQ ID NO:17) or a cytosine (C) or thymine (T) is at position 27 of SEQ ID NO:50 and SEQ ID NO:51, respectively; is predictive of survival in a subject.

Described herein are methods that comprise identifying the residue corresponding to single nucleotide polymorphism (SNP) rs716175, rs937793, rs3862432, rs3930162, rs17263706, rs3862434, rs8033595, rs12915189, rs7498042, rs12901137, rs12910489, rs12914286, rs7403002, rs11857476, rs7403440, rs10438448, or rs4344687 in the subject, the identification of SNP rs716175, rs937793, rs3862432, rs3930162, rs17263706, rs3862434, rs8033595, rs12915189, rs7498042, rs12901137, rs12910489, rs12914286, rs7403002, rs11857476, rs7403440, rs10438448, or rs4344687 is predictive of survival in the subject. For example, and not to be limiting, the method can comprise hybridizing the sample of nucleic acid from the subject with a probe, wherein the probe hybridizes under stringent conditions to an oligonucleotide consisting of SEQ ID NO:18 (C allele) or SEQ ID NO:19 (T allele), but does not hybridizes under stringent conditions to an oligonucleotide consisting of an A or G allele, wherein hybridization of the probe under stringent conditions to the nucleic acid from the subject is predictive of the likelihood of survival.

As used herein “allele” such as “C allele” is meant to refer to the SNP residue on either the sense or antisense strand. Thus, reference to “C allele” can refer to either strand and is therefore also a disclosure of “G allele” on the opposite strand.

The methods for predicting the likelihood of survival of a subject comprising determining the presence of one or more single nucleotide polymorphisms (SNPs) with an LOD score of greater than 3.5 with modulated expression of the IQGAP1 gene can further comprise a) obtaining a sample from a subject at a first time point; b) obtaining a second sample from the same subject at a second time point; c) determining the level of expression of one or more genes for each of the time points, wherein the one or more genes is CDC42, CORO1A, AURKB, CBX5, IQGAP1, TERF2, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1; d) predicting the likelihood of survival of the subject by comparing the expression level of one or more of the genes at the first time point to the expression level of one or more of the genes at the second time point, wherein a change in the expression level of one or more of the genes is predictive of survival. In one aspect an increase in the expression of CDC42 or TERF2 indicates a decreased likelihood of survival. In another aspect an increase in the expression of CORO1A, AURKB, CBX5, IQGAP1, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1 indicates an increased likelihood of survival. In another aspect, a decrease in the expression of CDC42 or TERF2 indicates an increased likelihood of survival. In yet another aspect, a decrease in the expression of CORO1A, AURKB, CBX5, IQGAP1, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1 indicates a decreased likelihood of survival.

In one aspect, the method for predicting the likelihood of survival of a subject comprising determining the presence of one or more single nucleotide polymorphisms (SNPs) with an LOD score of greater than 3.5 with modulated expression of the IQGAP1 gene can still further comprise determining telomere length of the subject; and correlating the telomere length with survival with telomere length in an age matched population of the subject. In one aspect the telomere length is the average telomere length. The telomere length can be determined by polymerase chain reaction performed on a sample from the subject. The sample can be, but is not limited to blood or lymphoid cells. More specifically, in one aspect, the lymphoid cells can comprise T cells.

2. Modulating Survival

Also described herein are methods method of modulating the risk of mortality in a subject comprising: administering an agonist or antagonist of one or more genes, wherein the one or more genes is CDC42, CORO1A, AURKB, CBX5, IQGAP1, TERF2, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, PRIVI1 or Cdc42GAP.

In one aspect, the described methods comprise decreasing the risk of mortality in a subject comprising administering an antagonist of CDC42 or TERF2. The antagonist can be a chemical, a compound, a small molecule, an inorganic molecule, an organic molecule, a drug, a protein, a cDNA, an aptamer, a peptide, an antibody, a morpholino, a triple helix molecule, an siRNA, an shRNAs, an miRNA, an antisense nucleic acid or a ribozyme that decreases the expression or activity of CDC42 or TERF2.

In another aspect, the methods comprise decreasing the risk of mortality in a subject comprising administering an agonist of CORO1A, AURKB, CBX5, IQGAP1, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1. The agonist can be a chemical, a compound, a small molecule, an inorganic molecule, an organic molecule, a drug, a protein, a cDNA, an aptamer, a peptide, an antibody, a morpholino, a triple helix molecule, an siRNA, an shRNAs, an miRNA, an antisense nucleic acid or a ribozyme that increases the expression or activity of CORO1A, AURKB, CBX5, IQGAP1, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1.

It is understood that the methods described herein are not limited to administration of one composition, as a combination of two, three, four, five or six compositions, can be administered, wherein each composition comprises a chemical, a compound, a small molecule, an inorganic molecule, an organic molecule, a drug, a protein, a cDNA, an aptamer, a peptide, an antibody, a morpholino, a triple helix molecule, an siRNA, an shRNAs, an miRNA, an antisense nucleic acid or a ribozyme that modulates the expression or activity of CDC42, TERF2, CORO1A, AURKB, CBX5, IQGAP1, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1.

i. Antibodies

Described herein are antibodies that specifically bind to the genes or gene products, proteins and fragments thereof described herein. The antibody can be a polyclonal antibody or a monoclonal antibody. The antibody can selectively bind a polypeptide. By “selectively binds” or “specifically binds” is meant an antibody binding reaction which is determinative of the presence of the antigen (in the present case, a polypeptide of a gene set forth herein or antigenic fragment thereof among a heterogeneous population of proteins and other biologics). Thus, under designated immunoassay conditions, the specified antibodies bind preferentially to a particular peptide and do not bind in a significant amount to other proteins in the sample. Preferably, selective binding includes binding at about or above 1.5 times assay background and the absence of significant binding is less than 1.5 times assay background.

Also described herein are antibodies that compete for binding to natural interactors or ligands to the proteins encoded by the genes set forth herein. In other words, the antibodies that disrupt interactions between the proteins of the genes set forth herein and their binding partners. For example, an antibody of the present invention can compete with a protein for a binding site (e.g. a receptor) on a cell or the antibody can compete with a protein for binding to another protein or biological molecule, such as a nucleic acid that is under the transcriptional control of a gene set forth herein. The antibody optionally can have either an antagonistic or agonistic function.

In one aspect he antibody binds a polypeptide in vitro, ex vivo or in vivo. Optionally, the antibody of the invention is labeled with a detectable moiety. For example, the detectable moiety can be selected from the group consisting of a fluorescent moiety, an enzyme-linked moiety, a biotin moiety and a radiolabeled moiety. The antibody can be used in techniques or procedures such as diagnostics, screening, or imaging. Anti-idiotypic antibodies and affinity matured antibodies are also considered to be part of the invention.

As used herein, the term “antibody” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. In one aspect, the “humanized” antibody is a human version of the antibody produced by a germ line mutant animal. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, the present invention provides a humanized version of an antibody, comprising at least one, two, three, four, or up to all CDRs of a monoclonal antibody that specifically binds to a protein or fragment thereof encoded by a gene set forth herein. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody can comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also can comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Peptides that inhibit expression are also described herein. Peptide libraries can be screened utilizing the screening methods set forth herein to identify peptides that inhibit expression of any of the genes or gene products set forth herein. These peptides can be derived from a protein that binds to any of the genes or gene products set forth herein. These peptides can be any peptide in a purified or non-purified form, such as peptides made of D-and/or L-configuration amino acids (in, for example, the form of random peptide libraries; see Lam et al., Nature 354:82-4, 1991), phosphopeptides (such as in the form of random or partially degenerate, directed phosphopeptide libraries; see, for example, Songyang et al., Cell 72:767-78, 1993).

ii. Antisense Nucleic Acids

Generally, the term “antisense” refers to a nucleic acid molecule capable of hybridizing to a portion of an RNA sequence (such as mRNA) by virtue of some sequence complementarity. The antisense nucleic acids described herein can be oligonucleotides that are double-stranded or single-stranded, RNA or DNA or a modification or derivative thereof, which can be directly administered to a cell (for example by administering the antisense molecule to the subject), or which can be produced intracellularly by transcription of exogenous, introduced sequences (for example by administering to the subject a vector that includes the antisense molecule under control of a promoter).

Antisense nucleic acids are polynucleotides, for example nucleic acid molecules that are at least 6 nucleotides in length, at least 10 nucleotides, at least 15 nucleotides, at least 20 nucleotides, at least 100 nucleotides, at least 200 nucleotides, such as 6 to 100 nucleotides. However, antisense molecules can be much longer. In particular examples, the nucleotide is modified at one or more base moiety, sugar moiety, or phosphate backbone (or combinations thereof), and can include other appending groups such as peptides, or agents facilitating transport across the cell membrane (Letsinger et al., Proc. Natl. Acad. Sci. USA 1989, 86:6553-6; Lemaitre et al., Proc. Natl. Acad. Sci. USA 1987, 84:648-52; WO 88/09810) or blood-brain barrier (WO 89/10134), hybridization triggered cleavage agents (Krol et al., BioTechniques 1988, 6:958-76) or intercalating agents (Zon, Pharm. Res. 5:539-49, 1988). Additional modifications include those set forth in U.S. Pat. Nos. 6,608,035, 7,176,296; 7,329,648; 7,262,489, 7,115,579; and 7,105,495.

Examples of modified base moieties include, but are not limited to: 5-fluorouracil, 5-bromouracil, 5-chlorouracil, 5-iodouracil, hypoxanthine, xanthine, acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl)uracil, and 2,6-diaminopurine.

Examples of modified sugar moieties include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In a particular example, an antisense molecule is an cc-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-41, 1987). The oligonucleotide can be conjugated to another molecule, such as a peptide, hybridization triggered cross-linking agent, transport agent, or hybridization-triggered cleavage agent. Oligonucleotides can include a targeting moiety that enhances uptake of the molecule by host cells. The targeting moiety can be a specific binding molecule, such as an antibody or fragment thereof that recognizes a molecule present on the surface of the host cell.

In a specific example, antisense molecules that recognize a nucleic acid set forth herein, include a catalytic RNA or a ribozyme (for example see WO 90/11364; WO 95/06764; and Sarver et al., Science 247:1222-5, 1990). Conjugates of antisense with a metal complex, such as terpyridylCu (II), capable of mediating mRNA hydrolysis, are described in Bashkin et al. (Appl. Biochem Biotechnol. 54:43-56, 1995). In one example, the antisense nucleotide is a 2′-0-methylribonucleotide (Inoue et al., Nucl. Acids Res. 15:6131-48, 1987), or a chimeric RNA-DNA analogue (Inoue et al., FEBS Lett. 215:327-30, 1987).

Antisense molecules can be generated by utilizing the Antisense Design algorithm of Integrated DNA Technologies, Inc. (1710 Commercial Park, Coralville, Iowa 52241 USA; (http://www.idtdna.com/Scitools/Applications/AntiSense/Antisense.aspx/).

iii. shRNA

shRNA (short hairpin RNA) is a DNA molecule that can be cloned into expression vectors to express siRNA (typically 19-29 nt RNA duplex) for RNAi interference. shRNA can have the following structural features: a short nucleotide sequence ranging from about 19-29 nucleotides derived from the target gene, followed by a short spacer of about 4-15 nucleotides (i.e. loop) and about a 19-29 nucleotide sequence that is the reverse complement of the initial target sequence.

iv. siRNA

Short interfering RNAs (siRNAs), also known as small interfering RNAs, are double-stranded RNAs that can induce sequence-specific post-transcriptional gene silencing, thereby decreasing gene expression (See, for example, U.S. Pat. Nos. 6,506,559, 7,056,704, 7,078,196, 6,107,094, 5,898,221, 6,573,099, and European Patent No. 1.144,623, all of which are hereby incorporated in their entireties by this reference). siRNas can be of various lengths as long as they maintain their function. In some examples, siRNA molecules are about 19-23 nucleotides in length, such as at least 21 nucleotides, for example at least 23 nucleotides. In one example, siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends. The direction of dsRNA processing determines whether a sense or an antisense target RNA can be cleaved by the produced siRNA endonuclease complex. Thus, siRNAs can be used to modulate expression of a gene set forth herein The effects of siRNAs have been demonstrated in cells from a variety of organisms, including Drosophila, C. elegans, insects, frogs, plants, fungi, mice and humans (for example, WO 02/44321; Gitlin et al., Nature 418:430-4, 2002; Caplen et al., Proc. Natl. Acad. Sci. 98:9742-9747, 2001; and Elbashir et al., Nature 411:494-8, 2001).

Utilizing sequence analysis tools, one of skill in the art can design siRNAs to specifically target any gene set forth herein for decreased gene expression. siRNAs that inhibit or silence gene expression can be obtained from numerous commercial entities that synthesize siRNAs, for example, Ambion Inc. (2130 Woodward Austin, Tex. 78744-1832, USA), Qiagen Inc. (27220 Turnberry Lane, Valencia, Calif. USA) and Dharmacon Inc. (650 Crescent Drive, #100 Lafayette, Colo. 80026, USA). The siRNAs synthesized by Ambion Inc., Qiagen Inc. or Dharmacon Inc, can be readily obtained from these and other entities by providing a GenBank Accession No. for the mRNA of any gene set forth herein. In addition, siRNAs can be generated by utilizing Invitrogen's BLOCK-IT™ RNAi Designer https://rnaidesigner.invitrogen.com/rnaiexpress.

v. Morpholinos

Morpholinos are synthetic antisense oligos that can block access of other molecules to small (about 25 base) regions of ribonucleic acid (RNA). Morpholinos are often used to determine gene function using reverse genetics methods by blocking access to mRNA. Morpholinos, usually about 25 bases in length, bind to complementary sequences of RNA by standard nucleic acid base-pairing. Morpholinos do not degrade their target RNA molecules. Instead, Morpholinos act by “steric hindrance”, binding to a target sequence within an RNA and simply interfering with molecules which might otherwise interact with the RNA. Morpholinos have been used in mammals, ranging from mice to humans.

Bound to the 5′-untranslated region of messenger RNA (mRNA), Morpholinos can interfere with progression of the ribosomal initiation complex from the 5′ cap to the start codon. This prevents translation of the coding region of the targeted transcript (called “knocking down” gene expression). Morpholinos can also interfere with pre-mRNA processing steps, usually by preventing the splice-directing snRNP complexes from binding to their targets at the borders of introns on a strand of pre-RNA. Preventing U1 (at the donor site) or U2/U5 (at the polypyrimidine moiety & acceptor site) from binding can cause modified splicing, commonly leading to exclusions of exons from the mature mRNA. Targeting some splice targets results in intron inclusions, while activation of cryptic splice sites can lead to partial inclusions or exclusions. Targets of U11/U12 snRNPs can also be blocked. Splice modification can be conveniently assayed by reverse-transcriptase polymerase chain reaction (RT-PCR) and is seen as a band shift after gel electrophoresis of RT-PCR products. Methods of designing, making and utilizing morpholinos are described in U.S. Pat. No. 6,867,349 which is incorporated herein by reference in its entirety.

vi. Small Molecules

Any small molecule that modulates expression, either directly or indirectly, of a gene or gene product described herein, can be utilized in the methods described herein to modulate the risk of mortality. These molecules can be identified in the scientific literature, in the StarLite database available from the European Bioinformatics Institute, in DrugBank (Wishart et al. Nucleic Acids Res. 2006 Jan. 1; 34 (Database issue):D668-72), package inserts, brochures, chemical suppliers (for example, Sigma, Tocris, Aurora Fine Chemicals, to name a few), or by any other means, such that one of skill in the art makes the association between a gene or gene product described herein and modulation of the expression of this gene or gene product, either direct or indirect, by a molecule.

The small molecules can be used therapeutically in combination with a pharmaceutically acceptable carrier. By “pharmaceutically acceptable” is meant a material that is not biologically or otherwise undesirable, i.e., the material can be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

vii. Administration

The described compounds and compositions, such as an antagonist or an agonist, can be administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like. Additional formulations that are suitable for other modes of administration include suppositories and, in some cases, through a buccal, sublingual, intraperitoneal, intravaginal, anal or intracranial route.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required can vary from subject to subject, depending on the species, age, weight and general condition of the subject, the particular composition used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect of modulating survival. The dosage should not be so large as to cause adverse side effects, such as unwanted cross-reactions, anaphylactic reactions, and the like. Generally, the dosage can vary with the age, condition, sex of the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

3. Screening Methods

Also described herein are methods of screening for compositions that modulate the expression of CDC42, CORO1A, AURKB, CBX5, IQGAP1, TERF2, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, PRIVI1, or the complement thereof, and at least one of the SNPs is rs716175, rs937793, rs3862432, rs3930162, rs17263706, rs3862434, rs8033595, rs12915189, rs7498042, rs12901137, rs12910489, rs12914286, rs7403002, rs11857476, rs7403440, rs10438448, or rs4344687.

Methods of screening for compositions that modulate gene expression are well known in the art. In one aspect the method can comprise a) contacting a composition with a gene or gene product of CDC42, CORO1A, AURKB, CBX5, IQGAP1, TERF2, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, PRIVI1, or the complement thereof, b) detecting binding of the compound to the gene or gene product; and c) associating binding with a modulation of expression and therefore a modulation of survival. This method can further comprise optimizing a compound that binds the gene product in an assay, for example, a cell based assay, an in silico assay, or an in vivo assay, that determines the functional ability to modulate expression.

4. Array

Also described herein is an array of nucleic acid molecules attached to a solid support for use in detecting the genes and single nucleotide polymorphisms (SNPs) described herein. Thus, described is an array of nucleic acid molecules attached to a solid support, wherein at least one of the nucleic acids comprise a sequence corresponding to genes CDC42, CORO1A, AURKB, CBX5, IQGAP1, TERF2, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, PRIVI1, or the complement thereof, and at least one of the SNPs is rs716175, rs937793, rs3862432, rs3930162, rs17263706, rs3862434, rs8033595, rs12915189, rs7498042, rs12901137, rs12910489, rs12914286, rs7403002, rs11857476, rs7403440, rs10438448, or rs4344687.

An array is an orderly arrangement of samples, providing a medium for matching known and unknown DNA samples based on base-pairing rules and automating the process of identifying the unknowns. An array experiment can make use of common assay systems such as microplates or standard blotting membranes, and can be created by hand or make use of robotics to deposit the sample. In general, arrays are described as macroarrays or microarrays, the difference being the size of the sample spots.

Microarrays contain sample spot sizes of about 300 microns or larger and can be easily imaged by existing gel and blot scanners. The sample spot sizes in microarray can be 300 microns or less, but typically less than 200 microns in diameter and these arrays usually contains thousands of spots. Microarrays require specialized robotics and/or imaging equipment that generally are not commercially available as a complete system. Terminologies that have been used in the literature to describe this technology include, but not limited to: biochip, DNA chip, DNA microarray, GeneChip® (Affymetrix, Inc which refers to its high density, oligonucleotide-based DNA arrays), and gene array.

A DNA microarray is a collection of microscopic DNA spots attached to a solid surface, such as glass, plastic or silicon chip forming an array for the purpose of expression profiling, monitoring expression levels for thousands of genes simultaneously. DNA microarrays, or DNA chips are fabricated by high-speed robotics, generally on glass or nylon substrates, for which probes with known identity are used to determine complementary binding, thus allowing massively parallel gene expression and gene discovery studies. An experiment with a single DNA chip can provide information on thousands of genes simultaneously. It is herein contemplated that the described microarrays can be used to monitor gene expression, disease diagnosis, gene discovery, drug discovery (pharmacogenomics), and toxicological research or toxicogenomics.

The affixed DNA segments are generally known as probes, thousands of which can be placed in known locations on a single DNA microarray. Microarray technology evolved from Southern blotting, whereby fragmented DNA is attached to a substrate and then probed with a known gene or fragment. Measuring gene expression using microarrays is relevant to many areas of biology and medicine, such as studying treatments, disease, and developmental stages. For example, microarrays can be used to identify disease genes by comparing gene expression in diseased and normal cells.

There are two variants of the DNA microarray technology, in terms of the property of arrayed DNA sequence with known identity. Type I microarrays comprise a probe cDNA (500˜5,000 bases long) that is immobilized to a solid surface such as glass using robot spotting and exposed to a set of targets either separately or in a mixture. This method is traditionally referred to as DNA microarray. With Type I microarrays, localized multiple copies of one or more polynucleotide sequences, preferably copies of a single polynucleotide sequence are immobilized on a plurality of defined regions of the substrate's surface. A polynucleotide refers to a chain of nucleotides ranging from 5 to 10,000 nucleotides. These immobilized copies of a polynucleotide sequence are suitable for use as probes in hybridization experiments.

Type II microarrays comprise an array of oligonucleotides (20˜80-mer oligos) or peptide nucleic acid (PNA) probes that is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc., which sells its photolithographically fabricated products under the GeneChip® trademark.

The basic concept behind the use of Type II arrays for gene expression is simple: labeled cDNA or cRNA targets derived from the mRNA of an experimental sample are hybridized to nucleic acid probes attached to the solid support. By monitoring the amount of label associated with each DNA location, it is possible to infer the abundance of each mRNA species represented. Although hybridization has been used for decades to detect and quantify nucleic acids, the combination of the miniaturization of the technology and the large and growing amounts of sequence information, have enormously expanded the scale at which gene expression can be studied.

In spotted microarrays (or two-channel or two-colour microarrays), the probes are oligonucleotides, cDNA or small fragments of PCR products corresponding to mRNAs. This type of array is typically hybridized with cDNA from two samples to be compared (e.g., patient and control) that are labeled with two different fluorophores. The samples can be mixed and hybridized to one single microarray that is then scanned, allowing the visualization of up-regulated and down-regulated genes in one go. The downside of this is that the absolute levels of gene expression cannot be observed, but only one chip is needed per experiment. One example of a provider for such microarrays is Eppendorf with their DualChip® platform.

In oligonucleotide microarrays (or single-channel microarrays), the probes are designed to match parts of the sequence of known or predicted mRNAs. There are commercially available designs that cover complete genomes from companies such as GE Healthcare, Affymetrix, Ocimum Biosolutions, or Agilent. These microarrays give estimations of gene expression and therefore the comparison of two conditions requires the use of two separate microarrays.

Long Oligonucleotide Arrays are composed of 60-mers, or 50-mers and are produced by ink-jet printing on a silica substrate. Short Oligonucleotide Arrays are composed of 25-mer or 30-mer and are produced by photolithographic synthesis (Affymetrix) on a silica substrate or piezoelectric deposition (GE Healthcare) on an acrylamide matrix. More recently, Maskless Array Synthesis from NimbleGen Systems has combined flexibility with large numbers of probes. Arrays can contain up to 390,000 spots, from a custom array design. New array formats are being developed to study specific pathways or disease states for a systems biology approach.

Oligonucleotide microarrays often contain control probes designed to hybridize with RNA spike-ins. The degree of hybridization between the spike-ins and the control probes is used to normalize the hybridization measurements for the target probes.

SNP microarrays are a particular type of DNA microarrays that are used to identify genetic variation in individuals and across populations. Short oligonucleotide arrays can be used to identify the single nucleotide polymorphisms (SNPs) that are thought to be responsible for genetic variation and the source of susceptibility to genetically caused diseases. Generally termed genotyping applications, DNA microarrays may be used in this fashion for forensic applications, rapidly discovering or measuring genetic predisposition to disease, or identifying DNA-based drug candidates.

These SNP microarrays are also being used to profile somatic mutations in cancer, specifically loss of heterozygosity events and amplifications and deletions of regions of DNA. Amplifications and deletions can also be detected using comparative genomic hybridization in conjunction with microarrays.

Resequencing arrays have also been developed to sequence portions of the genome in individuals. These arrays may be used to evaluate germline mutations in individuals, or somatic mutations in cancers.

Genome tiling arrays include overlapping oligonucleotides designed to blanket an entire genomic region of interest. Many companies have successfully designed tiling arrays that cover whole human chromosomes.

Samples may be any sample containing polynucleotides (polynucleotide targets) of interest and obtained from any bodily fluid (blood, urine, saliva, phlegm, gastric juices, etc.), cultured cells, biopsies, or other tissue preparations. DNA or RNA can be isolated from the sample according to any of a number of methods well known to those of skill in the art. For example, methods of purification of nucleic acids are described in Laboratory Techniques in Biochemistry and Molecular Biology: Hybridization With Nucleic Acid Probes. Part I. Theory and Nucleic Acid Preparation, P. Tijssen, ed. Elsevier (1993). In one embodiment, total RNA is isolated using the TRIzol total RNA isolation reagent (Life Technologies, Inc., Rockville, Md.) and RNA is isolated using oligo d(T) column chromatography or glass beads. After hybridization and processing, the hybridization signals obtained should reflect accurately the amounts of control target polynucleotide added to the sample.

Some of the key elements of selection and design are common to the production of all microarrays, regardless of their intended application. Strategies to optimize probe hybridization, for example, are invariably included in the process of probe selection. Hybridization under particular pH, salt, and temperature conditions can be optimized by taking into account melting temperatures and using empirical rules that correlate with desired hybridization behaviors.

To obtain a complete picture of a gene's activity, some probes are selected from regions shared by multiple splice or polyadenylation variants. In other cases, unique probes that distinguish between variants are favored. Inter-probe distance is also factored into the selection process.

A different set of strategies is used to select probes for genotyping arrays that rely on multiple probes to interrogate individual nucleotides in a sequence. The identity of a target base can be deduced using four identical probes that vary only in the target position, each containing one of the four possible bases.

Alternatively, the presence of a consensus sequence can be tested using one or two probes representing specific alleles. To genotype heterozygous or genetically mixed samples, arrays with many probes can be created to provide redundant information, resulting in unequivocal genotyping. In addition, generic probes can be used in some applications to maximize flexibility. Some probe arrays, for example, allow the separation and analysis of individual reaction products from complex mixtures, such as those used in some protocols to identify single nucleotide polymorphisms (SNPs).

The plurality of defined regions on the substrate can be arranged in a variety of formats. For example, the regions may be arranged perpendicular or in parallel to the length of the casing. Furthermore, the targets do not have to be directly bound to the substrate, but rather can be bound to the substrate through a linker group. The linker groups may typically vary from about 6 to 50 atoms long. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probes.

Sample polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, biotin, and the like.

Labeling can be carried out during an amplification reaction, such as polymerase chain reaction and in vitro or in vivo transcription reactions. Alternatively, the labeling moiety can be incorporated after hybridization once a probe-target complex his formed. In one preferred embodiment, biotin is first incorporated during an amplification step as described herein. After the hybridization reaction, unbound nucleic acids are rinsed away so that the only biotin remaining bound to the substrate is that attached to target olynucleotides that are hybridized to the polynucleotide probes. Then, an avidin-conjugated fluorophore, such as avidin-phycoerythrin, that binds with high affinity to biotin is added.

Hybridization causes a polynucleotide probe and a complementary target to form a stable duplex through base pairing. Hybridization methods are well known to those skilled in the art Stringent conditions for hybridization can be defined by salt concentration, temperature, and other chemicals and conditions. Varying additional parameters, such as hybridization time, the concentration of detergent (sodium dodecyl sulfate, SDS) or solvent (formamide), and the inclusion or exclusion of carrier DNA, are well known to those skilled in the art. Additional variations on these conditions will be readily apparent to those skilled in the art (Wahl, G. M. and S. L. Berger (1987) Methods Enzymol. 152:399-407; Kimmel, A. R. (1987) Methods Enzymol. 152:507-511; Ausubel, F. M. et al. (1997) Short Protocols in Molecular Biology, John Wiley & Sons, New York, N.Y.; and Sambrook, J. et al. (1989) Molecular Cloning, A Laboratory Manual, Cold Spring Harbor Press, Plainview, N.Y.).

Methods for detecting complex formation are well known to those skilled in the art. In a preferred embodiment, the polynucleotide probes are labeled with a fluorescent label and measurement of levels and patterns of complex formation is accomplished by fluorescence microscopy, preferably confocal fluorescence microscopy. An argon ion laser excites the fluorescent label, emissions are directed to a photomultiplier and the amount of emitted light detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensities. The scanned image is examined to determine the abundance/expression level of each hybridized target polynucleotide.

In a differential hybridization experiment, polynucleotide targets from two or more different biological samples are labeled with two or more different fluorescent labels with different emission wavelengths. Fluorescent signals are detected separately with different photomultipliers set to detect specific wavelengths. The relative abundances/expression levels of the target polynucleotides in two or more samples is obtained. Typically, microarray fluorescence intensities can be normalized to take into account variations in hybridization intensities when more than one microarray is used under similar test conditions. In one embodiment, individual polynucleotide probe/target complex hybridization intensities are normalized using the intensities derived from internal normalization controls contained on each microarray.

Microarray manufacturing can begin with a 5-inch square quartz wafer. Initially the quartz is washed to ensure uniform hydroxylation across its surface. Because quartz is naturally hydroxylated, it provides an excellent substrate for the attachment of chemicals, such as linker molecules, that are later used to position the probes on the arrays.

The wafer is placed in a bath of silane, which reacts with the hydroxyl groups of the quartz, and forms a matrix of covalently linked molecules. The distance between these silane molecules determines the probes' packing density, allowing arrays to hold over 500,000 probe locations, or features, within a mere 1.28 square centimeters. Each of these features harbors millions of identical DNA molecules. The silane film provides a uniform hydroxyl density to initiate probe assembly. Linker molecules, attached to the silane matrix, provide a surface that may be spatially activated by light.

Probe synthesis occurs in parallel, resulting in the addition of an A, C, T, or G nucleotide to multiple growing chains simultaneously. To define which oligonucleotide chains will receive a nucleotide in each step, photolithographic masks, carrying 18 to 20 square micron windows that correspond to the dimensions of individual features, are placed over the coated wafer. The windows are distributed over the mask based on the desired sequence of each probe. When ultraviolet light is shone over the mask in the first step of synthesis, the exposed linkers become deprotected and are available for nucleotide coupling.

Once the desired features have been activated, a solution containing a single type of deoxynucleotide with a removable protection group is flushed over the wafer's surface. The nucleotide attaches to the activated linkers, initiating the synthesis process.

Although each position in the sequence of an oligonucleotide can be occupied by 1 of 4 nucleotides, resulting in an apparent need for 25×4, or 100, different masks per wafer, the synthesis process can be designed to significantly reduce this requirement. Algorithms that help minimize mask usage calculate how to best coordinate probe growth by adjusting synthesis rates of individual probes and identifying situations when the same mask can be used multiple times.

Microarrays can be fabricated using a variety of technologies, including printing with fine-pointed pins onto glass slides, photolithography using pre-made masks, photolithography using dynamic micromirror devices, ink-jet printing (Lausted C, et al. Genome Biol. 2004;5(8):R58), or electrochemistry on microelectrode arrays.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides.

The substrate can be any substrate to which polynucleotide probes can be attached, including but not limited to glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734, which are hereby incorporated by reference for the teaching of making and using polynucleotide arrays. Commercially available polynucleotide arrays, such as Affymetrix GeneChip™, can also be used. Use of the GeneChip™ to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

Typical dispensers include a micropipette delivering solution to the substrate with a robotic system to control the position of the micropipette with respect to the substrate. There can be a multiplicity of dispensers so that reagents can be delivered to the reaction regions simultaneously. For example, a microarray can be formed by using ink-jet technology based on the piezoelectric effect, whereby a narrow tube containing a liquid of interest, such as oligonucleotide synthesis reagents, is encircled by an adapter. An electric charge sent across the adapter causes the adapter to expand at a different rate than the tube and forces a small drop of liquid onto a substrate (Baldeschweiler et al. PCT publication WO95/251116).

Thus, described is an array of nucleic acid molecules attached to a solid support, wherein at least one of the nucleic acids comprise a gene or a fragment thereof or a SNP described herein.

5. Hybridization/Selective Hybridization

In one aspect, the expression levels of the genes or SNPs described herein can be determined through the use of hybridization or selective hybridization. The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described herein to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 3, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein described for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered described herein.

It is understood that those of skill in the art understand that if a composition or method meets any one of these criteria for determining hybridization either collectively or singly it is a composition or method that is described herein.

6. Nucleic Acids

The described nucleic acids can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

The sequences for IQGAP1, including human IQGAP1, as well as other analogs, and alleles of these genes, and splice variants and other types of variants, are available in a variety of protein and gene databases, including Genbank. For example, a genomic sequence for human IQGAP1 is described in Accession No. NT_{—}010274.17. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information described herein and known in the art.

Also described are compositions including primers and probes, which are capable of interacting with the described nucleic acids. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the described primers hybridize with the described nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In some aspects, a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

7. Computer Readable Mediums

It is understood that the described nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein described. Specifically contemplated herein is the display of these sequences on computer readable mediums, such as, commercially available floppy disks, tapes, chips, hard drives, compact disks, and video disks, or other computer readable mediums. Also described are the binary code representations of the described sequences. Those of skill in the art understand what computer readable mediums. Thus, computer readable mediums on which the nucleic acids or protein sequences are recorded, stored, or saved. Thus, described are computer readable mediums comprising the sequences and information regarding the sequences set forth herein.

8. Kits

The materials described herein as well as other materials can be packaged together in any suitable combination as a kit useful for performing, or aiding in the performance of, the described method. It is useful if the kit components in a given kit are designed and adapted for use together in the described method. For example described are kits for detecting one or more of the SNPs described herin, the kit comprising, for example, nucleic acid probes that bind to a target nucleic acid having the one or more SNPs but not to a nucleic acid that does not comprise the one or more SNPs. The described kits can also include profiles of SNPs in control populations with instructions for interpreting the results.

9. Uses

The described compositions can be used in a variety of ways as research tools. Other uses are described, apparent from the disclosure, and/or will be understood by those in the art.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Gene Expression Profiles Associated with Aging and Mortality in Humans

The data from the expression levels for 2,151 always-expressed genes in the CEU cell lines was used to construct an estimate of biological age based on gene expression levels, then used for a biological age estimate in a proportional hazards model of survival after blood draw to assess the degree to which gene expression profiles can serve as biomarkers of aging and/or longevity. A multivariate survival model based on age-adjusted gene expression was used to predict mortality among the CEU grandparents. This approach was not specifically designed to identify heritable variants that affect longevity. Rather, this approach focused on stable variation in gene expressions that affect or mark longevity. The methods described herein address variation in gene expression that is anticipated from inherited genetic variants, including copy number variants.

i. Materials and Methods

The CEPH/Utah family resource originated from bloods drawn from 46 three-generation families, each consisting of 5-15 siblings, their two parents, and 2-4 grandparents who were still alive at the time of the family blood draws in the early 1980s.

Cheung et al. extracted RNA from transformed B lymphocytes obtained from the Coriell Cell Repository (http://locus.umdnj.edu/nigms/ceph/ceph.html) for a total of 247 CEU family members (124 male; 123 female), including 104 of the grandparents for whom there was survival data. Expression levels for 8793 probesets were measured using Affymetrix HG-Focus arrays. The resulting expression data were deposited in the NCBI Gene Expression Omnibus (GEO) database, accession numbers GSE1485 and GSE2552. Both datasets were combined to maximize the number of individuals available for study.

Each probeset on the HG-Focus array consisted of a set 11-20 pairs of probes, each consisting of a 25 mer oligonucleotide representing a “perfect match” to the target sequence and a “mismatch” probe made by substituting an alternative nucleotide at the 13^{th} position. To improve the fit of probesets to genes, the “HG-Focus RefSeq Transcript” mapping was used and was supplied by Liu, et al., available from http://gauss.dbb.georgetown.edu/liblab/affyprobeminer/transcript.html. After re-mapping, 8174 probesets were available for analysis.

Whether each gene was expressed beyond baseline was tested in each sample using the Wilcoxon signed rank test as described in the Affymetrix Microarray Suite version 5. This is a test for absolute “presence” vs. “absence,” testing if the observed signals for each probeset were significantly greater than background. For purposes of the present analysis, all probesets were eliminated from consideration that were not called “present” (p<0.04) or “marginal” (p<0.06) in each of the grandparents' samples. This step left 2,151 always-expressed genes.

Wu and Irizarry's GeneChip robust multiarray averaging (GCRMA) method was used to normalize all expression levels. The GCRMA and MAS 5.0 algorithms were implemented in the “affy” and “gcrma” packages available from the Bioconductor website (http://www.bioconductor.org).

All samples were evaluated for outlying observations in relation to average background, scale factor, number of genes called “present,” and 3′ to 5′ ratios for GAPDH, following the procedures described by Wilson and colleagues. Twelve samples were excluded from analysis because they were out-of-range for at least one test. In addition, 5 samples exhibited inappropriately high or low levels of expression of both RPS4Y1, encoded on the Y chromosome, and the X-inactivating sequence transcript (XIST), expressed only in women. Without exception, in women high expression of RPS4Y1 was coupled with low expression of XIST, and in men low expression of RPS4Y1 was coupled with high expression of XIST. Since all grandparents are by definition fertile, we could rule out sex chromosome abnormalities as an explanation. These samples were excluded on the grounds that they had been mistakenly attributed to the wrong person. After these exclusions, at least one sample from 238 individuals (including all 104 grandparents) remained.

Many CEU family members, including most of the grandparents, had expression data available from multiple arrays (usually two, although two of the grandparents had four arrays available). For these individuals, gcrma-corrected expression levels were averaged for each probeset prior to analysis.

a. Univariate Analyses

Two separate analyses were performed of expression as a function of age at draw. First, only the grandparents were considered, who were effectively unrelated to one another, although careful analysis of the records of the Utah Population Database has revealed that a few of the CEU grandparents are distantly related. The grandparents' expression data represented were treated as independent observations, and ordinary least squares methods were used to regress expression level for each of the always-expressed probesets against age, adjusting for sex.

Strong genetic correlations in expression level should be present within each three-generation CEU family. For this reason, standard linear regression approaches are not appropriate for the study of age-related variation in expression patterns. Instead, linear mixed-effects models were used to adjust for the kinship among family members. The substantially larger sample size (238 vs. 104) and wider range of ages at draw (5-97 vs. 57-97) led to the consideration of both linear and quadratic effects for age at draw in the three-generation families. Otherwise, the model fit was the same as for the grandparents: expression level was modeled as a function of age at draw, age squared, and sex. Heritability estimates were computed for 3-generation pedigrees using SOLAR.

b. Survival Models

Grandparents ranged in age from 57 to 97 years of age at the time of the blood draw. Median follow-up age was 84.7 years (range 65.7-100.8 years). Survival was measured from age at draw to age at death or follow-up. A proportional hazards model as used adjusting for sex, year of birth, and age at draw to test the association of each expression level with survival. Each proportional hazards model was tested for nonproportionality using the cox.zph function in the R software package (http://www.r-project.org). Although some nonproportionality was detected with p-values below 0.0001, none of the genes strongly associated with survival had a p-value for nonproportionality lower than 0.28 (EMP3).

c. Bivariate Age-at-Draw vs. Survival Models

Fisher's likelihood ratio test was computed of the composite null hypothesis that expression was unrelated to either aging or longevity. One thousand random permutations of the survival data was generated because of concerns that age at blood draw was not completely independent of survival after blood draw, even under the null hypothesis. Random permutations were generated by shuffling the rows of a matrix that included age at draw, age at follow-up, sex, and vital status as columns. For each iteration, the randomly ordered phenotypes were assigned to the unpermuted gene expression vectors, and computed the linear regressions on age at draw, the proportional hazards models and the Fisher test. This procedure allowed the correlation structure of the expression data, and the correlation structure of the survival data to remain intact, while testing the relationship between the two datasets.

d. Adjustment for Multiple Comparisons

Adjustments for multiple testing were appropriate in this context, but many methods masked hidden assumptions about the dependency structure of the data or the true proportion of false null hypotheses. Even permutation-based methods retained some vulnerability to hidden dependencies within microarray data. Therefore, a simple Bonferroni correction was employed in presenting the results of the univariate analyses, and a Monte Carlo permutation test as employed in presenting the results of the bivariate analyses.

e. Multivariate Analyses

To assess the relationship between age at draw and multiple expression levels, the least absolute shrinkage and selection operator (LASSO) algorithm of Tibshirani was employed to build a linear model of age at draw as a function of multiple expression levels. Only the grandparents' data was used to avoid complex dependency structures. Briefly, the LASSO approach minimizes the residual sum of squares in a multiple regression model subject to the constraint that the sum of the absolute values of the standardized coefficients is less than a specified constant. Efron, et al. showed that computing all possible LASSO models is feasible and provides a basis for rationally choosing among them, by minimizing Cₚ or by cross-validation.

Cross-validation procedures divide the data at random into K equal subsets, and, for i=1 to K, use all the data not in the ith subset to estimate the model, and the data in ith subset to test the model predictions. The goal is to find the value of the tuning parameter that minimizes the mean square prediction error across the K subsets. With relatively small datasets, however, K-fold cross-validation procedures are often unstable, and this proved to be the case with the present data set.

K=104, was set, which leads to the leave-one-out cross-validation procedure (LOOCV) in our sample of 104 grandparents. The resulting LOOCV curve were compared to curves generated from 100 random permutations of the data, performed as described herein. Comparing the cross-validation curve to a null distribution of cross-validation curves not only provided information on the optimal setting of the tuning parameter, but also on the probability that the result were due to chance.

For each value of the tuning parameter, corresponding to a step in which a predictor variable can be added or dropped, the model selected was used to predict each subject's age at draw. These “biological age” estimates, together with the subjects' actual age and sex, were used to predict age at death in a proportional hazards model.

The approach of Segal was followed, using the LASSO approach described herein, to regress the deviance residuals of a baseline proportional hazards regression (adjusted for sex and age at draw) against the set of expression levels. The permuted LOOCV approach described herein was used to identify optimal settings for the tuning parameter and to assess the probability that the observed pattern was the result of chance.

ii. Results

a. Changes in Gene Expression with Age: CEU Grandparents

If the expression of a gene responds to the progress of senescence by rising or falling over the adult lifespan, then expression differences among chronologically age-matched adults will reflect variation in rates of biological aging. In order to establish age-related changes in gene expression levels, expressions most strongly associated with age at blood draw were first identified. Of the 8,793 total measured expressions (probesets on Affymetrix HG-Focus arrays), only the 2,151 always-expressed genes (in all 362 cell lines and three generations of Utah CEU families) were used to examine the relationship between individual expression levels and age at draw in the CEU grandparent cell lines. Expression was modeled as a simple linear function of age at draw. Expression levels were reported on a log₂ scale, so that a linear increase or decrease in measured expression level corresponded to a multiplicative increase in gene expression. Because the grandparents were effectively unrelated to one another, no adjustment for kinship was necessary, and conventional linear regression models were used.

Of the 2,151 always-expressed genes, 345 (16%) expressions were associated with age at draw in the CEU grandparents at a nominal p<0.05. Of these, 125 increased with age and 220 decreased with age. Table 2 shows the magnitude, direction, and significance of the linear regression of expression as a function of age at draw for the top ten age-associated expression levels for the 104 CEU grandparents, after adjusting for sex. None of the always-expressed genes was linearly associated with age at draw at a nominal p-value below the Bonferroni 5% threshold of 2.3×10⁻⁵. The strongest association of expression level with age was CDC42 (cell division cycle 42), which exhibited strongly increased expression with age, with a sex-adjusted p-value of 3.1×10⁻⁵, and an unadjusted p-value of 1.3×10⁻⁵. Among the 10 most strongly age-associated expression levels (Table 2), equal numbers (5) increase and decrease with age.

Also shown in Table 2 is the estimated heritability of expression (H²) for each gene listed, and the correlation of expression between spouses. Most of the genes listed in Table 2 had heritabilities between 0.2 and 0.5. CORO1A had the highest estimated heritability (0.66), while expression levels for RNH1 and TMEM142C did not appear to be heritable. Spouse correlations were generally very low, with moderate positive correlations observed for CDC6 (0.28) and CORO1A (0.23).

TABLE 2
Top ten age-associated expression levels in CEU grandparents.

| HG-Focus | Gene | | | | Spouse |
|---|---|---|---|---|---|
| Probeset | Symbol | Z | p-value | H² | Correlation |
| HF6524 | CDC42 | 4.36 | 3.14E−05 | 0.26 | 0.062 |
| HF2432 | MKNK2 | 4.09 | 8.65E−05 | 0.35 | 0.12 |
| HF8737 | SH3BGRL | 4.07 | 9.51E−05 | 0.45 | −0.057 |
| HF4113 | RNH1 | −4.03 | 1.09E−04 | 0.00 | −0.010 |
| HF6098 | TMEM142C | 3.75 | 2.97E−04 | 0.09 | −0.13 |
| HF7682 | CDC6 | −3.73 | 3.13E−04 | 0.33 | 0.28 |
| HF1646 | USP1 | −3.65 | 4.23E−04 | 0.23 | 0.055 |
| HF1405 | EDF1 | 3.60 | 4.88E−04 | 0.27 | −0.028 |
| HF982 | QDPR | −3.60 | 5.01E−04 | 0.28 | 0.029 |
| HF7873 | CORO1A | −3.49 | 7.19E−04 | 0.66 | 0.23 |

Notes:
Probeset-probeset name from HG-Focus Refseq transcript library; Gene Symbol(s)-HUGO symbol name or names corresponding to current mapping of probeset sequence; Z-Z score of linear model of expression vs. age, adjusted for sex; p-value-probability of observing a Z greater than that observed under the null hypothesis; H2-heritability.

b. Changes in Gene Expression with Age: Three-Generation Families

Expression was modeled as a simple linear function of age at draw using all three generations of the CEU families. Out of the full set of 2,151 always-expressed genes, 784 (36.4%) expression levels showed age effects with p-values below the Bonferroni 5% threshold of 2.3×10⁻⁵. Of these, 348 increased with age, and 436 decreased with age. A larger number of age-related changes were observed when a quadratic term was added to the model, allowing for curvature in the regression of expression against age. A two degree-of-freedom test of significance of the combined linear and quadratic effects yielded 907 (42.2%) expression levels significantly associated with age at draw, allowing for multiple comparisons.

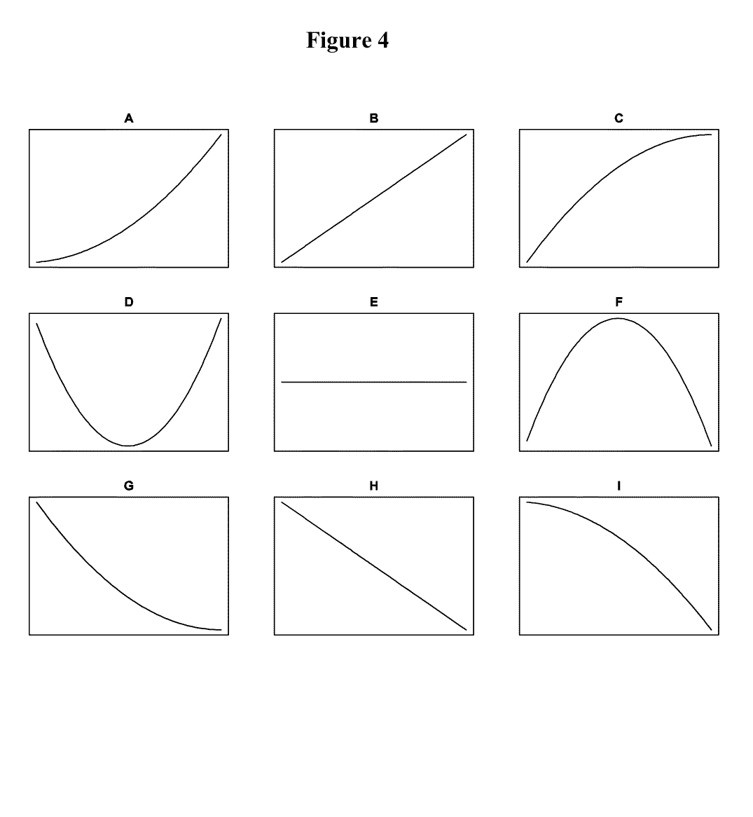

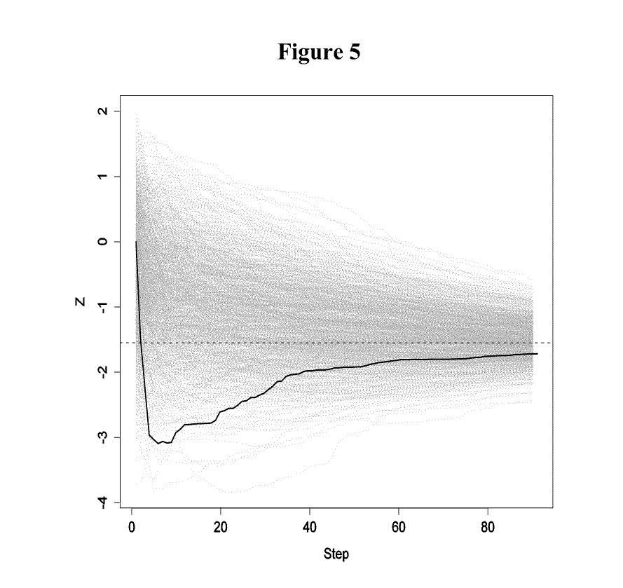

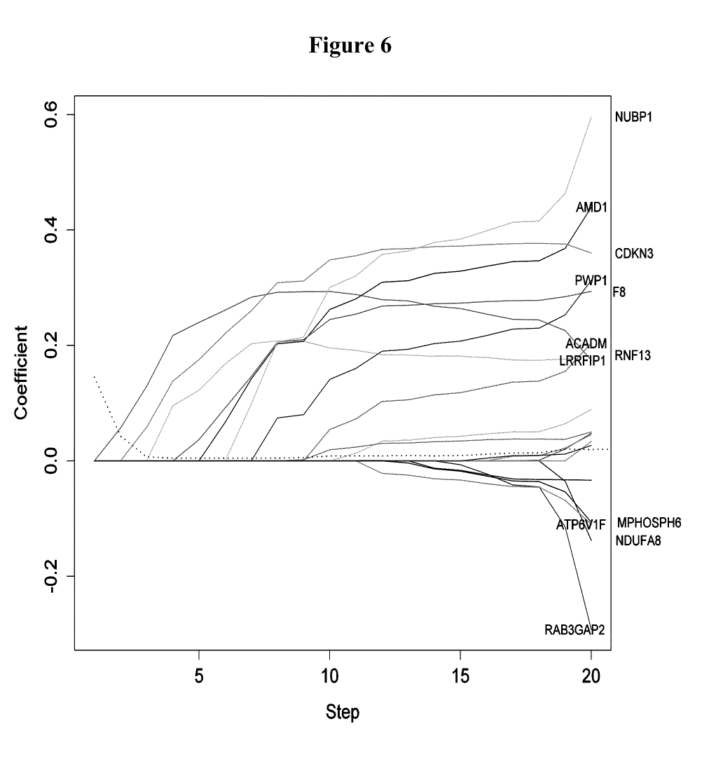

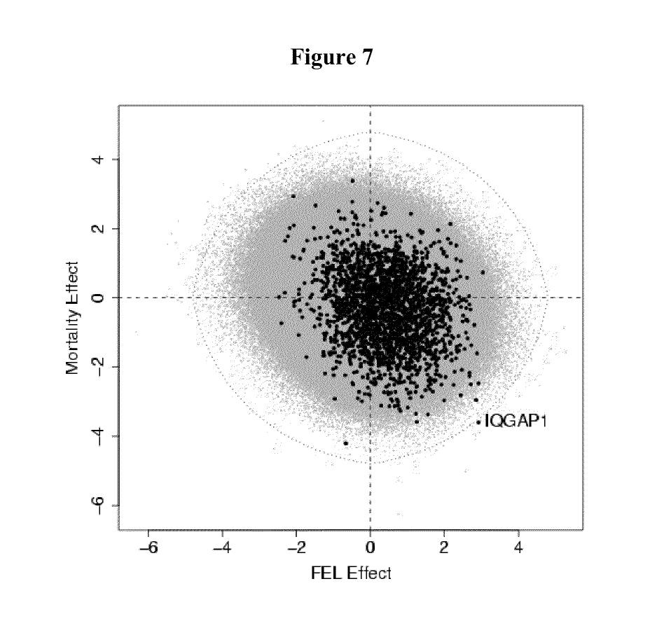

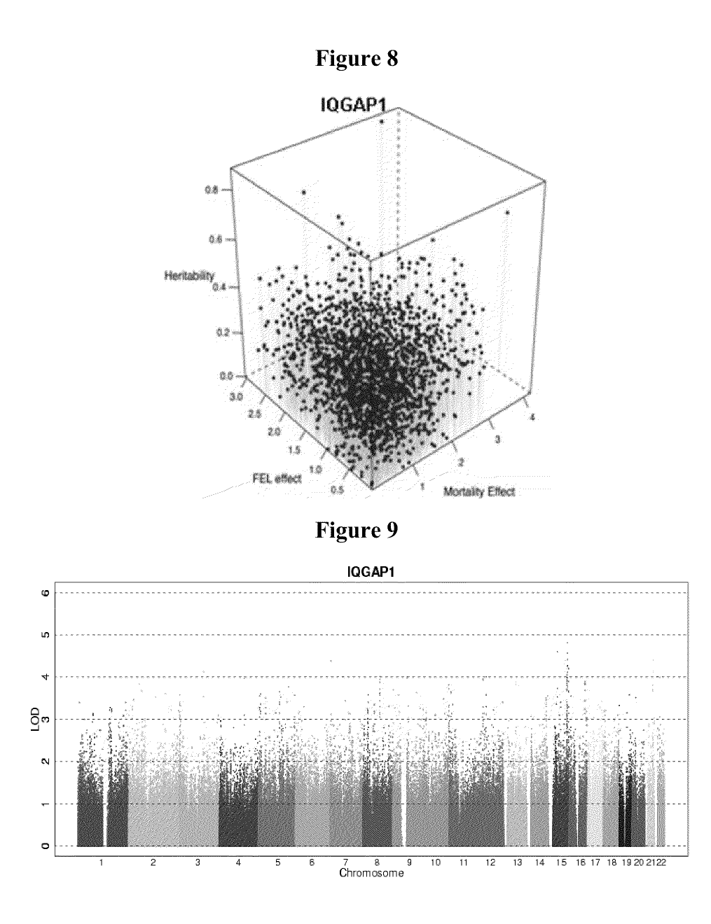

For comparative purposes, the shape of the relationship between age at draw and expression level was classified into nine categories, labeled A-I for convenience. The category definitions are listed in Table 3 and idealized representations of each are displayed in FIG. 4. More than half (1244; 57.8%) of the expression levels were not associated with age strongly enough to overcome the Bonferroni adjustment; of these, 443 (20.6%) exhibited no significant association with age at draw even at a nominal p-value of 0.05. Categories A (superlinear rise) and I (superlinear drop) had no members with p-values exceeding the Bonferroni threshold. The quadratic-only categories D (U-shaped) and F (inverted U) were rarely observed, with only 13 and 8 members, respectively. The expression levels were reported on a log₂ scale; hence, a linear increase (B) or decrease (H) in measured expression level corresponded to a multiplicative increase in gene expression, while a truly linear change in gene expression corresponded to a sublinear change (C or G) on a log scale.

TABLE 3
Categories of age-related changes in expression level observed in
2,151 always-expressed genes in three-generation CEU family data.

| | | Linear | Quadratic | | |
|---|---|---|---|---|---|
| Category | Shape | Effect | Effect | Count | Percent |
| Superlinear Rise | A | Positive | Positive | 0 | 0.0% |
| Linear Rise | B | Positive | Nonsig- | 148 | 6.9% |
| | | | nificant | | |
| Sublinear Rise | C | Positive | Negative | 257 | 11.9% |
| U-shaped | D | Nonsig- | Positive | 13 | 0.6% |
| | | nificant | | | |
| Unrelated to Age | E | Nonsig- | Nonsig- | 1244 | 57.8% |
| | | nificant | nificant | | |
| Inverted U | F | Nonsig- | Negative | 8 | 0.4% |
| | | nificant | | | |
| Sublinear Drop | G | Negative | Positive | 232 | 10.8% |
| Linear Drop | H | Negative | Nonsig- | 249 | 11.6% |
| | | | nificant | | |
| Superlinear Drop | I | Negative | Negative | 0 | 0.0% |

Table 4 lists the top 20 associations in the three-generation families in increasing order of p-value. 17 of the 20 strongest associations in Table 4, were negative overall (i.e., the regression slope of a model that omits the age² term is negative), and the shape category was either G (sublinear drop) or H (linear drop). Expression of SAFB and PSMD4 increased sublinearly with age, and expression of BAT2 increased linearly.

TABLE 4
Top twenty age-associated expression levels in three-generation families.

| Probeset | Z.lin | Z.age | Z.age₂ | Z.sex | Shape | p-value | Gene Symbol(s) |
|---|---|---|---|---|---|---|---|
| HG-FocusHF4679 | −15.14 | −7.83 | 4.22 | 1.42 | G | 2.47E−39 | PRKAR1A |
| HG-FocusHF9208 | −15.46 | −5.51 | 2.08 | 0.92 | G | 6.58E−38 | EIF3S10 |
| HG-FocusHF5720 | −14.27 | −7.27 | 4.27 | 0.71 | G | 1.25E−36 | SF3B1 |
| HG-FocusHF9350 | 14.53 | 7.51 | −3.97 | −0.93 | C | 1.85E−36 | SAFB |
| HG-FocusHF5595 | −14.31 | −6.78 | 3.49 | 2.23 | G | 1.27E−35 | RNF11 |
| HG-FocusHF9502 | −13.90 | −7.45 | 3.94 | −0.90 | G | 1.78E−34 | IFNA1, IFNA2, |
| | | | | | | | IFNA4, IFNA6, |
| | | | | | | | IFNA7, IFNA10, |
| | | | | | | | IFNA13, IFNA14, |
| | | | | | | | IFNA16, IFNA17 |
| HG-FocusHF5779 | −13.24 | −7.55 | 4.39 | −1.46 | G | 3.87E−33 | BCL10 |
| HG-FocusHF8192 | −12.65 | −8.40 | 5.40 | 0.55 | G | 4.88E−33 | MARCH7 |
| HG-FocusHF8737 | −11.45 | −9.90 | 7.01 | 0.18 | G | 1.69E−32 | SH3BGRL |
| HG-FocusHF3239 | 13.94 | 4.79 | −1.61 | −1.19 | B | 2.07E−32 | BAT2 |
| HG-FocusHF4119 | 13.04 | 6.76 | −3.92 | 0.15 | C | 2.55E−31 | PSMD4, |
| | | | | | | | PSMD4P2 |
| HG-FocusHF10285 | −13.52 | −4.71 | 1.56 | 1.28 | H | 9.53E−31 | SEC24B |
| HG-FocusHF1562 | −12.56 | −6.69 | 3.92 | 1.03 | G | 2.21E−30 | TANK |
| HG-FocusHF10040 | −13.81 | −1.71 | −1.25 | 0.48 | H | 6.67E−30 | SFRS2IP |
| HG-FocusHF1673 | −13.22 | −4.23 | 1.29 | 0.81 | H | 8.14E−30 | SMNDC1 |
| HG-FocusHF2428 | −12.83 | −5.85 | 2.85 | 0.70 | G | 8.28E−30 | MARCKS |
| HG-FocusHF1383 | −12.91 | −5.76 | 2.95 | 0.71 | G | 8.97E−30 | AGL |
| HG-FocusHF6922 | −11.40 | −8.35 | 5.81 | 0.99 | G | 1.74E−29 | HNRPH1 |
| HG-FocusHF1269 | −13.10 | −3.58 | 0.66 | 1.95 | H | 3.82E−29 | C1D |
| HG-FocusHF2499 | −12.70 | −5.18 | 2.34 | 0.67 | G | 7.27E−29 | VPS4B |

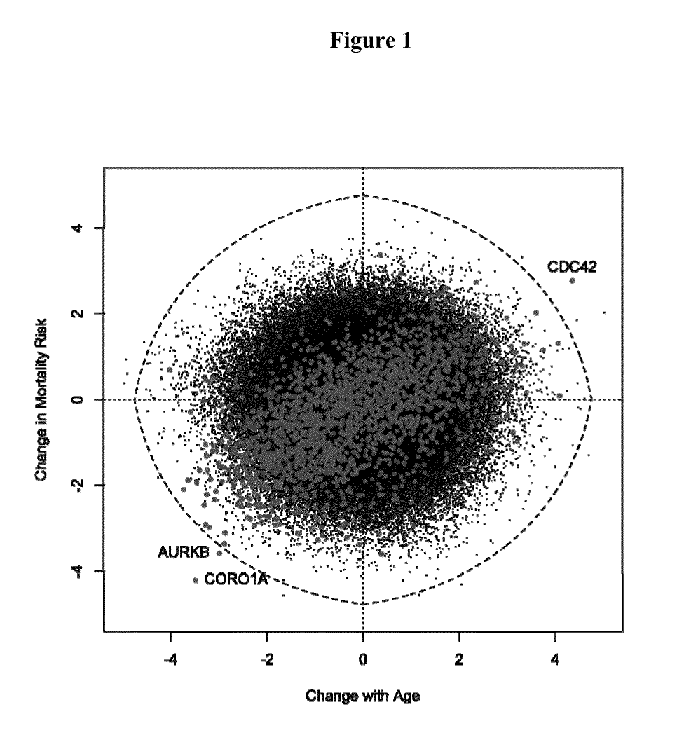

As a note to Table 4: Probeset—probeset name from HG-Focus Refseq transcript library; Z.lin—Z score of linear model of expression vs. age, adjusted for sex; Z.age—Z score of linear term of linear+quadratic model of expression vs. age, adjusted for sex; Z.age²—Z score of quadratic term in linear+quadratic model; Shape—relationship between age and expression, as defined in Table 2 and FIG. 1; p-value—probability of observing a χ² [2 d.f.] greater than the observed likelihood ratio test for the linear+quadratic model of expression vs. age; Gene Symbol(s)—HUGO symbol name or names corresponding to current mapping of probeset sequence.

This analysis of changes in gene expression with age over all three generations of CEU families revealed a large number of highly significant associations. In general, the pattern of age-related changes observed among the grandparents alone, reported in Table 6 was quite different from the pattern observed across all three generations. The correlation coefficient between the linear-only Z score for three generations and the linear Z score for grandparents-only was −0.09. However, interpretation of age-related changes in expression across three generations, ages 5-97, was difficult because senescence can be confounded with sexual or physiological maturation, as well as secular trends occurring over such a long span of donor birth years in these families.

c. Proportional Hazards Models of Survival vs. Gene Expression

Gene expressions most strongly associated with survival were also tested for, independently of their associations with age at blood draw. Table 5 shows the ten strongest associations between age-adjusted expression and survival after blood draw among the 104 CEU grandparents. None of the observed effects exceeded the Bonferroni threshold of 2.3×10⁻⁵, although CORO1A (coronin) came close. Nine of the ten strongest associations of age-adjusted expression with mortality were negative, meaning that relative overexpression of the gene was associated with reduced mortality. Interestingly, the one exception was TERF2IP (telomeric repeat binding factor 2, interacting protein), which is thought to protect telomeric DNA from nonhomologous end-joining. Note that only one gene (CORO1A) appeared in both Tables 2 and 5, supporting the notion that gene expressions strongly associated with age at blood draw were not necessarily strongly associated with survival, too. As in Table 2, most of the estimated heritabilities in Table 5 were 0.25 or greater, while CORO1A was again highest (0.66). No evidence for heritability of expression was seen for TERF2IP, KIF2C, or EMP3. Moderately strong positive spouse correlations were observed for IQGAP1 (0.22) and CORO1A (0.23).

TABLE 5
Top 10 survival-associated expression levels in CEU grandparents.

| HG-Focus | Gene | | | | Spouse |
|---|---|---|---|---|---|
| Probeset | Symbol | Z | p-value | H² | Correlation |
| HF7873 | CORO1A | −4.20 | 2.64E−05 | 0.66 | 0.23 |
| HF8664 | IQGAP1 | −3.60 | 3.19E−04 | 0.59 | 0.22 |
| HF6054 | AURKB | −3.58 | 3.41E−04 | 0.34 | −0.04 |
| HF7038 | TERF2IP | 3.37 | 7.45E−04 | 0.064 | 0.0089 |
| HF6482 | CBX5 | −3.37 | 7.46E−04 | 0.41 | −0.058 |
| HF8349 | KIF2C | −3.35 | 8.02E−04 | 0.046 | 0.035 |
| HF657 | ACTR2 | −3.27 | 1.07E−03 | 0.45 | 0.068 |
| HF2735 | SPAG5 | −3.21 | 1.31E−03 | 0.39 | −0.012 |
| HF2854 | MTF2 | −3.17 | 1.54E−03 | 0.25 | 0.040 |
| HF7574 | EMP3 | −3.13 | 1.74E−03 | 0.068 | −0.0026 |

As a note to Table 5: Probeset-probeset name from HG-Focus Refseq transcript library; Gene Symbol(s)-HUGO symbol name or names corresponding to current mapping of probeset sequence; Z-Z score from proportional hazards model of survival vs. age-adjusted expression; p-value-probability of observing a Z greater than that observed; H²-heritability.

A total of 167 (7.8%) expression levels were associated with survival in the CEU grandparents at a nominal p<0.05. Of these, 48 were associated with age at blood draw at a nominal p<0.05.

d. Combining Information on Age at Draw and Survival after Draw

Both the association of age with expression level, and the association of expression level with survival after blood draw, contain distinct and important information about the relationship of gene expression to aging in humans. Two strategies were used to combine these pieces of information: 1) a test of the joint null hypothesis that expression was related to neither age nor survival; and 2) a two-stage model that constructed a multivariate estimator of biological age, then used it to predict survival.

e. Tests of the Joint Hypotheses that Expression is Related to neither Age nor Survival

FIG. 1 shows the relationship between Z scores for age effects and survival effects on (age-adjusted) expression levels, for the CEU grandparents. Using Fisher's likelihood ratio approach, the observed Z scores (large red dots) were compared to those generated by a 10% sample of 1000 random permutations of the phenotypic (age, sex, and survival) data, while keeping the expression vectors constant (small black dots). The dashed ellipse was drawn at the fiftieth largest χ² value observed for the 2151 always-expressed genes and 1000 permutations. The results are shown this way to approximate the fifth percentile of the null distribution, adjusted for 2151 comparisons. Three genes fell outside the threshold: CORO1A (0%), CDC42 (0.2%), and AURKB (aurora kinase B; 1.5%). Expression of CDC42 increased with age among the CEU grandparents, and higher age-adjusted expression was associated with higher mortality. CORO1A and AURKB represented the opposite extreme of this same pattern: expression decreased with age, and higher age-adjusted expression was associated with lower mortality.

Overall there was a fairly strong positive correlation (r=0.51; p<2.2×10⁻¹⁶) between Z scores for age-related expression change and age-adjusted survival in FIG. 1. The orientation of this general pattern was described by the contrast between CORO1A in the lower left quadrant, and CDC42 in the upper right quadrant. Points located in the lower left quadrant (e.g., CORO1A) represented expression levels that decreased with age, and where relative underexpression was associated with higher mortality. Points located in the upper right quadrant (e.g., CDC42) represented expression levels that increased with age, and where relative overexpression was also associated with higher mortality. In contrast, the randomly permuted data were distributed roughly equally around the origin, including many points in the upper left and lower right quadrants. The observed distribution included relatively few points in these quadrants, and none near the extremes of the null distribution.

f. Multivariate Models of Biological Age vs Survival

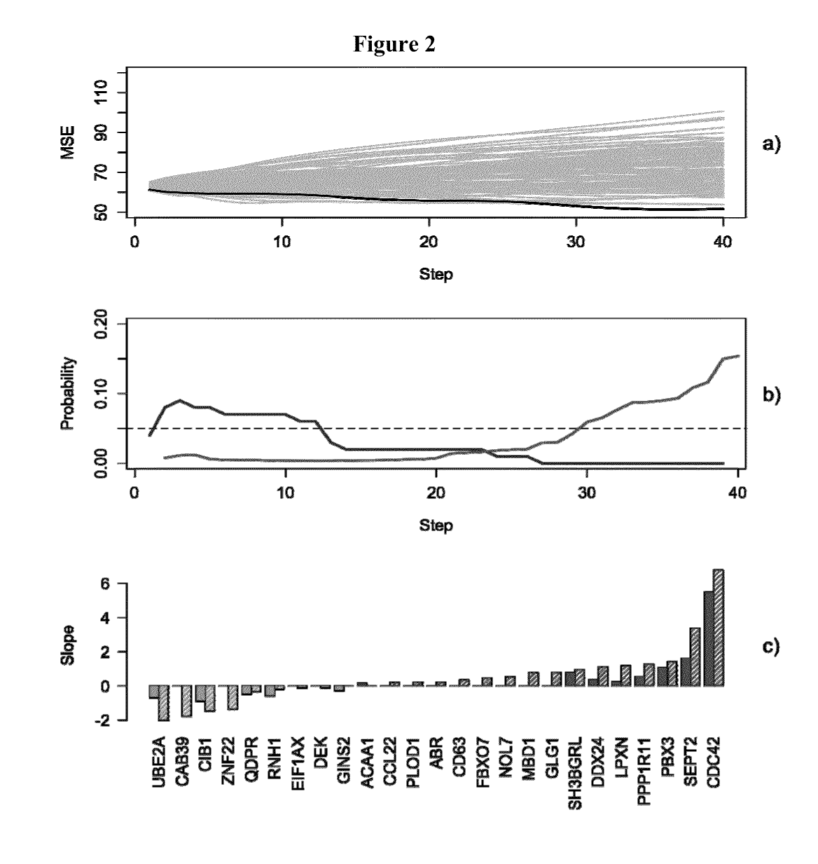

In the Methods section, estimation and cross-validation procedures were described for the least absolute shrinkage and selection operator (LASSO) model of biological age. FIG. 2a shows the leave-one-out cross-validation (LOOCV) curve observed over the first 40 steps (black line), compared to LOOCV curves generated under 100 random permutations of the phenotypic data. In FIG. 2b the blue line plots the probability, at each step, that a model generated from random data has a mean squared error (MSE) as low as the observed model; the red line plots p-values generated by proportional hazards regression using the biological age estimate as a predictor of mortality. It was clear from FIGS. 2a and 2b that the observed LOOCV curve was below the fifth percentile of the distribution of random curves by step 14, and the observed curve remain lower than any randomly generated curve for all steps after step 28. Meanwhile, the predicted biological age generated from the observed model was strongly significantly related to survival after blood draw from steps 2 to 30. The estimated MSE at step 14 was 57, corresponding to a prediction error of ±7.6 years (7.4 years at step 28). FIG. 2c shows the slope coefficients at steps 14 and 28.

The most parsimonious model with an MSE lower than 95% of simulations occurred at step 14. Coefficients of the model, in decreasing order of absolute value, are: CDC42 (5.5), SEPT2 (1.6), PBX3 (1.1), CIB1 (−0.91), SH3BGRL (0.77), UBE2A (−0.71), RNH1 (−0.60), PPP1R11 (0.55), QDPR (−0.48), DDX24 (0.36), GINS2 (−0.30), LPXN (0.24), and ACAA1 (0.16). The positive association of CDC42 with age at draw dominated this model. This remained true at steps 20, 40, 60, and 80 (data not shown). Expression levels of CDC42, SH3BGRL, QDPR, and RNH1 were also among the 10 most strongly associated with age at draw for CEU grandparents in the univariate analysis reported in Table 2. In the leave-one-out cross-validation models, all the selected terms were included over 85% of the time at step 14, with the exception of LPXN (39%), DDX24 (4%), and ACAA1 (0%).

The step 14 model from FIG. 2c was used to generate estimated biological ages for the CEU grandparents, which were then included together with chronological age at draw and sex, in a proportional hazards model of survival. As expected, predicted biological age was positively associated with mortality. The hazard rate ratio (HRR, an estimate of relative risk) for a single year increased in estimated biological age was 1.33 (95% Confidence Interval: 1.10-1.62).

An alternative approach to modeling survival as a function of estimated biological age would be to model it as a function of the difference between biological and chronological age. This is equivalent to forcing both biological age and chronological age to have the same slope (with opposite signs), and is efficient only if both variables are scaled identically. This approach was evaluated by rescaling biological age to have 0 mean and unit variance, then multiplying by the standard deviation of chronological age (7.9) and adding the mean chronological age (71.5); that technique produced results (not shown here) essentially identical to our original method, reported herein.

g. Multivariate Models of Survival vs. Expression Level

The analysis herein demonstrated that biological age, estimated from gene expression levels that change with age, was a significant predictor of remaining life span. An important potential weakness of this analysis was that it placed too much emphasis on gene expressions that vary systematically with age in cross-sectional data.

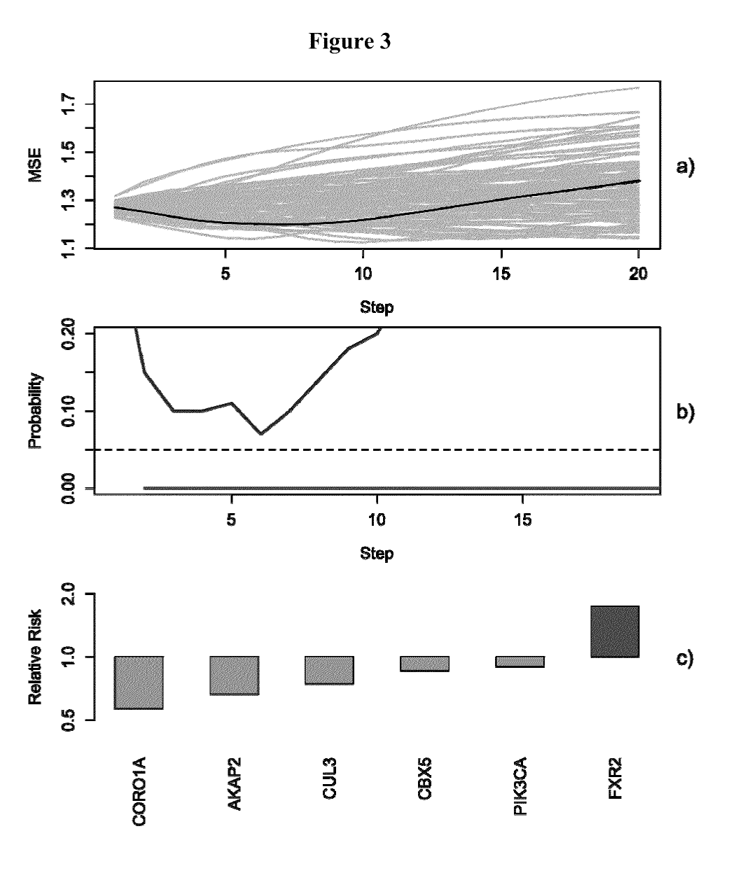

In an effort to circumvent this limitation, the LASSO approach was also applied to model survival as a direct multivariate function of expression levels. Deviance residuals were computed from a baseline survival model adjusted for age at draw and sex, and LASSO was used to identify expression levels associated with variation in the deviance residual (see Methods). The same permuted LOOCV approach was used for cross-validation and permutation tests of significance (described herein in Methods). Results are shown in FIG. 3.

FIG. 3a shows the cross-validation results compared to results of 100 random permutations of the phenotype data; a minimum was reached at step 7. In FIG. 3b, the observed cross-validation MSE was smaller than 94% of those observed in permuted data at step 7. Model coefficients are in order of decreasing absolute value: CORO1A (−0.27), FXR2 (0.21), CBX5 (−0.074), PIK3CA (−0.0094), AKAP2 (−0.0086), and CUL3 (−0.0081). The model was dominated by the positive association between FXR2 expression and mortality, and the negative association between CORO1A expression and mortality. A negative association between CBX5 and mortality also contributed. The effects of the other three genes are an order of magnitude smaller. Table 6 shows that the linear predictors generated by this model were strongly associated with survival: p-value=4.0×10⁻8; inter-quartile relative risk (IQRR)=2.35; median estimated survival difference=5.5 years. Predicted mortality from the model accounted for 23% of the variation (R²=0.23) in survival among the CEU grandparents. Model coefficients for individual genes were converted into interquartile relative risks in FIG. 3c and plotted on a log scale.

As a note to Table 6: IQRR—relative risk comparing the 75^{th} percentile of estimated risk (0.21) to the 25^{th} percentile (−0.03), adjusted for actual age and sex; Median Survival 1^{st} quartile—estimated median survival for subjects at the 25^{th} percentile of estimated risk (adjusted for age and sex); Median Survival 4^{th} quartile—estimated median survival for subjects at the 75^{th} percentile of estimated risk (adjusted for age and sex).

The nominal p-value given in Table 6 for the overall data was very low, given that the same survival data were used for estimation and testing. Evaluating the ability to predict survival in selected subgroups (e.g., males vs. females, for various causes of death, or varying numbers of years after blood draw) was more informative than showing how well the model fits the overall data. Table 6 shows that the model predicts similar mortality risks for males and females. Since age was the single largest risk factor for multiple life-threatening diseases, a biomarker that truly reflects biological age (or rate of aging) will be associated with risks of dying from not one, but several common causes of death due to age-related diseases. Therefore, the panel of gene expressions from the survival model (LASSO) was tested for associations with mortality risks for the common causes of death. Table 6 shows that the LASSO model predicted risk from multiple causes of death, in spite of very small sample sizes, with a particularly strong effect for deaths attributed to diabetes. The number of causes of death listed in Table 6 (6) was larger than the number of genes contributing importantly to the model (3), so the possibility that these associations were caused by overfitting seems slight.

TABLE 6
Performance of LASSO mortality model by sex, cause of death, and time
since blood draw.

| | | | | | | Median | Median |
|---|---|---|---|---|---|---|---|
| | | | | | | Survival | Survival |
| | At | | | | | 1st | 4th |
| Subset | Risk | Deaths | IQRR | Z | p-value | Quartile | Quartile |
| All | 104 | 72 | 2.35 | 5.49 | 4.0E−08 | 89.3 | 83.8 |
| Males | 52 | 40 | 2.73 | 3.76 | 0.00017 | 90.5 | 81.7 |
| Females | 52 | 32 | 2.31 | 3.99 | 6.6E−05 | 88.8 | 84.7 |

Cause of Death

| | | | | | |
|---|---|---|---|---|---|
| Heart | 104 | 19 | 2.17 | 2.65 | 0.0080 |
| Cancer | 104 | 14 | 2.37 | 2.14 | 0.032 |
| Stroke | 104 | 11 | 3.73 | 3.54 | 0.00040 |
| Diabetes | 104 | 5 | 7.72 | 3.21 | 0.0013 |
| Inf/Pneu | 104 | 5 | 3.48 | 2.49 | 0.013 |
| Cognitive | 104 | 10 | 2.57 | 2.12 | 0.034 |

Years after Blood Draw

| | | | | | | | |
|---|---|---|---|---|---|---|---|
| 1 | 103 | 71 | 2.35 | 5.46 | 4.8E−08 | 89.1 | 82.6 |
| 3 | 95 | 64 | 2.27 | 4.75 | 2.0E−06 | 89.4 | 84.6 |
| 5 | 88 | 57 | 2.53 | 4.27 | 2.0E−05 | 90.4 | 85.2 |
| 10 | 72 | 41 | 2.41 | 3.30 | 0.00097 | 91.5 | 88.1 |

Table 6 also shows that the LASSO model remained strongly predictive of mortality for at least 10 years following blood draw. Thus, these associations were not likely due to the presence of terminal diseases in some research subjects at the time of enrollment in the study.

Although Table 6 demonstrates that the predicted model was not strongly affected by subgroup influences on gene expression, a more robust assessment of whether the fit of the model was produced by chance was given by the permutation distribution of cross-validation curves shown in FIG. 3b. The minimum cross-validation MSE of the model was smaller than 94% of those generated by random permutation of the phenotype data (step 7). Therefore, there was approximately a 6% probability that the LASSO model of survival, based on 2151 measures of gene expression, fit the data this well by chance.

h. Environmental Exposures

Inter-individual differences in gene expression profiles in the Utah CEU lymphoblastoid cell lines reflect not only heritable genetic influences, but also environmental exposures experienced at any time prior to blood draw. Therefore, the possibility that gene expressions associated with survival may simply reflect exposures (or non-exposure) to a common toxic agent, such as cigarette smoke, was considered. The available data did not contain information on environmental exposures; however, affiliation with the Church of Jesus Christ of Latter-day Saints (or LDS church), available from the Utah Population Database, indirectly provided information about exposures that affect mortality risks. Merrill, et al., using data from the 1996 statewide Utah Health Status Survey, reported that 9.2% of LDS men (vs. 24.5% of non-LDS Utah men) reported being current smokers, while only 4.1% of LDS women reported smoking (vs. 23.1% of non-LDS women). Of the 104 grandparents with expression data who linked to the UPDB, 77 (74%) were strongly affiliated with the LDS church, and this was probably an underestimate because UPDB data were very incomplete in this regard. It was thus expected that only a small number (probably less than 10) of the grandparents were smokers. In previous work with the Utah Population Database, it was shown that reduced smoking and alcohol consumption among active church members probably accounted for 1.3 additional years of life expectancy compared to Utahans unaffiliated or inactive in the church. Inclusion of church affiliation as a covariate in the survival models slightly strengthened the relationship of the model predictions to survival (data not shown), indicating that the results reported in Table 6 were not confounded by smoking. Furthermore, none of the genes listed in Table 5, or included in the LASSO survival model, have been reported to be significantly affected by cigarette smoking.

It was apparent in Tables 2 and 5 that spouse correlations for some individual gene expressions were moderately high. Across the entire set of 2,151 always-expressed genes, the highest observed spouse correlation was 0.50 (PRSS3). CORO1A and IQGAP1 exhibited spouse correlations>0.2, although the estimated heritability for each was quite high. The mean spouse r² across the entire dataset was 0.962 while the maximum r² for 100 random pairs of grandparents was slightly smaller (0.958; minimum=0.952, mean=0.955). Overall, then, spouse expression profiles were slightly more strongly correlated than expected by chance, although the level of correlation among all expression profiles was very high. However, the correlation of mortality risk between spouses (using the deviance residuals from the baseline proportional hazards model—see Methods) was only 0.075 (p-value 0.45), so correlations in expression were not likely to confound the survival analysis.

iii. Discussion

While FIG. 1 shows clearly that, in general, the intensity and direction of age-related change in expression of a gene among the CEU grandparents was related to the strength of association of that gene's expression with survival, identifying individual genes that are most strongly related to aging is less simple. A variety of approaches to this task were taken, and several genes appeared to be important in more than one context. In particular, CDC42 and CORO1A appeared to be associated with both age at draw and survival after blood draw, whether univariate or multivariate approaches were applied. CDC42 expression increased with increasing age among the CEU grandparents, and, after adjusting for age, higher expression of CDC42 was associated with higher mortality. CDC42 was also the dominant factor in our multivariate model of biological age, which is a significant predictor of mortality.

Coronin (CORO1A) is an actin-binding protein with potentially important functions in both T cell-mediated immunity and mitochondrial apoptosis. Shiow, et al. reported that coronin defects in mice cause peripheral T cell deficiency, and described a human patient with severe combined immunodeficiency who had mutations in both coronin alleles. A nonsense mutation in CORO1A was recently shown to suppress autoimmune response in a mouse model of systemic lupus erythematosus, further suggesting that coronin is critical to immune functioning. Moreover, the inadvertent coronin knockout mice of Haralds son, et al. show substantially decreased mitochondrial membrane potential and increased apoptosis in T cells, but not B cells.

Aurora-B kinase (AURKB) is a key member of the chromosomal passenger complex which is critical in the regulation and conduct of mitosis. Inhibition of AURKB in tumor cells leads to growth inhibition and apoptosis. CBX5 encodes the human HP1α heterochromatin protein, importantly involved in the construction and maintenance of chromatin and hence an important regulator of gene expression. CBX5 expression decreased with age in the CEU grandparents, and reduced expression was associated with greater mortality. Likewise, reduced expression of IQGAP1 is associated with increased mortality, although expression of IQGAP1 is not strongly related to age. IQGAP1 is an effector of CDC42, and is involved in multiple signaling pathways. Goring, et al. reported a LOD score for cis-regulation of IQGAP1 expression of 5.8 (chromosome 15, 99 cM). Similarly, it was found that, in the 60 CEU grandparents genotyped by the International HapMap Projects (www.hapmap.org), several single nucleotide polymorphisms (SNPs) near the IQGAP1 gene were strongly associated with IQGAP1 expression. Thus, the region immediately surrounding IQGAP1 harbors genetic variants associated with variation in human survival.

Overexpression of TERF2 interacting protein (TERF2IP, aka hRAP1) in lymphoblastoid cell lines was associated with increased mortality; however, increased expression of TERF2IP should lead to increased telomere length, which has been associated with decreased mortality in the CEU grandparents. Unlike IQGAP1, variation in TERF2IP expression was not highly heritable, either in transformed (H²=0.09; p=0.10 in our data) or untransformed (H²=0.15; p=0.054 in Goring et al., 2007) lymphocytes. TERF2IP expression was uncorrelated with subjects' telomere lengths as measured in whole blood (r=0.04). This was a consequence of the cell transformation process, which activates telomerase so that cell lines may grow indefinitely in culture; possibly variable TERF2IP expression was marking some variation in telomerase activity in transformed lymphocytes that was indirectly related to longevity. A recent report links longer telomeres to increased risk of breast cancer. Among the CEU grandparents, however, TERF2IP expression was not significantly associated with cancer mortality risk.

Some striking patterns of association of gene expression with age and mortality have been described, based on lymphoblastoid cell lines derived from ordinary blood samples, and stored for years as a replenishable source of DNA for genetic studies. Thus, frozen cell lines also have considerable value as sources of phenotypic information on transcription, translation, and other cellular processes helpful in predicting the future health of the donors.

### 2. Example 2

### Association of Gene Expression Patterns in Lymphoblastoid Cell Lines with Familial Longevity and Survival

The association of familial excess longevity (FEL) with patterns of gene expression was investigated in a set of lymphoblastoid cell lines derived from 104 donors who were members of the CEPH Utah families (CEU). Previously, it was observed that gene expression was strongly associated with age and mortality in these individuals. Using data from the Utah Population Database, the FEL, the kinship-weighted mean difference between observed and expected lifespan among relatives, was estimated and the association of FEL with individual and grouped gene expression vs. survival data was tested. In general, FEL was negatively correlated with the hazard rate associated with gene expression: genes associated with increased risk of death in the proband were associated with decreased FEL in the relatives, and genes associated with decreased risk of death were associated with increased FEL. Overall the correlation was −0.56 (p-value 2.2×10−16). Individual genes strongly associated with both survival and FEL include IQGAP1 and AURKB. Individual genes strongly associated with age at draw and FEL include CDC42, ORC2L, and PSAT1.

i. Materials and Methods

a. Cell Lines

The CEPH/Utah family resource originated from bloods drawn from 46 three-generation families, each consisting of 5-15 siblings, their two parents, and 2-4 grandparents who were still alive at the time of the family blood draws in the early 1980s. Cheung et al. extracted RNA from transformed B lymphocytes obtained from the Coriell Cell Repository (http://locus.umdnj.edu/nigms/ceph/ceph.html) for a total of 247 CEU family members (124 male; 123 female), including 104 of the grandparents that had associated survival data.

b. Microarray Data

Expression levels for 8793 probesets were measured using Affymetrix HG-Focus arrays. The resulting expression data were deposited in the NCBI Gene Expression Omnibus (GEO) database, accession numbers GSE1485 and GSE2552. Both datasets were combined to maximize the number of individuals available for study. Each probeset on the HG-Focus array consisted of a set 11-20 pairs of probes, each consisting of a 25 mer oligonucleotide representing a “perfect match” to the target sequence and a “mismatch” probe made by substituting an alternative nucleotide at the 13th position. Several recent studies have shown that the original mapping of probes and probesets to genes, based on the human UniGene Build 133 database, contains many errors. To improve the fit of probesets to genes, the “HG-Focus RefSeq Transcript” mapping supplied by Liu, et al., and available from http://gauss.dbb.georgetown.edu/liblab/affyprobeminer/transcript.html, as used. After re-mapping, 8174 probesets were available for analysis. Whether each gene was expressed beyond baseline in each sample was tested using the Wilcoxon signed rank test as described in the Affymetrix Microarray Suite version 5. This is a test for absolute “presence” vs. “absence,” testing if the observed signals for each probeset were significantly greater than background. For purposes of the present analysis, all probesets that were not called “present” (p<0.04) or “marginal” (p<0.06) in each of the grandparents' samples were eliminated from consideration. This step left 2,151 always-expressed genes.

c. Array Normalization and QC

Three different microarray normalization approaches RMA, GCRMA, and MAS5.0), were evaluated by comparing mean heritabilities of 100 randomly selected genes. Wu and Irizarry's GeneChip robust multiarray averaging (GCRMA) yielded the highest mean H2. The GCRMA and MAS 5.0 algorithms that were used were implemented in the “affy” and “gcrma” packages available from the Bioconductor website (http://www.bioconductor.org). All samples were evaluated for outlying observations in relation to average background, scale factor, number of genes called “present”, and 3′ to 5′ ratios for GAPDH, following the procedures described by Wilson et al. Twelve samples were excluded from analysis because they were out-of-range for at least one test. In addition, 5 samples exhibited inappropriately high or low levels of expression of both RPS4Y1, encoded on the Y chromosome, and the X-inactivating sequence transcript (XIST), expressed only in women. Without exception, in women high expression of RPS4Y1 was coupled with low expression of XIST, and in men low expression of RPS4Y1 was coupled with high expression of XIST. Since all grandparents are by definition fertile, sex chromosome abnormalities as an explanation were ruled out. These samples were excluded on the grounds that they had been mistakenly attributed to the wrong person. After these exclusions, at least one sample from 238 individuals (including all 104 grandparents) remained. Many CEU family members, including most of the grandparents, had expression data available from multiple arrays (usually two, although two of the grandparents had four arrays available). For these individuals, gcrma-corrected expression levels for each probeset were averaged prior to analysis.

d. Demographic and Genealogical Data

Follow-up data on the 104 grandparents are summarized in Table 7. Ninety-two of the subjects could be connected to one or more biological relatives born prior to 1915 and followed until at least age 65, so that familial excess longevity (FEL, see Kerber, et al. 2001) could be computed. FEL is a kinship-weighted average of the excess longevity (age at death or censoring minus expected age at death) among a subject's relatives.

e. Statistical Methods

Univariate comparisons of FEL vs. gene expression were computed by linear regression and summarized as Z scores. Univariate comparisons of survival vs. gene expression were computed by proportional hazards regression and summarized by Z scores. Bivariate comparisons of gene expression vs. FEL and longevity were evaluated with Fisher's likelihood ratio chi-square statistic, but null distributions were computed over 1,000 permutations of the demographic and FEL data. Multivariate models of FEL were estimated using Tibshirani's LASSO method, and compared to LASSO models computed on the 1,000 random permutations described herein.

ii. Results

All of the top 20 genes (ranked by increasing p-value and shown in Table 7) were positively associated with FEL. None of the univariate associations were significant after adjusting for multiple comparisons. Noteworthy genes on this list include CDC42EP3, and IQGAP1, which interact with CDC42.

iii. Discussion

Studying the association of gene expression patterns with familial longevity as well as all-cause mortality is helpful in identifying genes and eQTLs involved in modulating rates of aging in human populations. The combination of familial and individual information examined here yielded several associations: 1) although IQGAP1 was the only individual gene expression associated with both decreased mortality and increased familial longevity beyond what could be expected by chance in this small sample, there was a well-defined pattern of association in the expected direction between the effects of expression on mortality and the effects of expression on familial longevity; 2) IQGAP1 and CDC42EP3 are both effectors of CDC42, a gene previously shown to be strongly associated with both age at draw and all-cause mortality; and 3) a fairly simple multivariate model of FEL consisting of a linear combination of 5 gene expression values was a strongly significant predictor of all-cause mortality.

TABLE 7
Top 20 Univariate Associations of Expression with FEL

| Symbol | Z | p-value |
|---|---|---|
| CDKN3 | 3.31 | 0.0013 |
| PBK | 3.26 | 0.0016 |
| PDXK | 3.18 | 0.0020 |
| AMD1 | 3.17 | 0.0021 |
| RNF13 | 3.02 | 0.0033 |
| CDC42EP3 | 2.98 | 0.0037 |
| F8 | 2.94 | 0.0041 |
| LRRFIP1 | 2.84 | 0.0056 |
| SHOC2 | 2.83 | 0.0058 |
| GNE | 2.79 | 0.0064 |
| IQGAP1 | 2.78 | 0.0066 |
| RNPEP | 2.78 | 0.0067 |
| PDIA5 | 2.75 | 0.0073 |
| HEXB | 2.69 | 0.0084 |
| ZDHHC13 | 2.68 | 0.0087 |
| RAD51AP1 | 2.66 | 0.0094 |
| GGH | 2.64 | 0.0097 |
| CETN3 | 2.64 | 0.0098 |
| GFPT1 | 2.60 | 0.0109 |
| PRIM1 | 2.58 | 0.0115 |

What is claimed is:

1. A method for predicting the likelihood of survival of a subject comprising:
a) obtaining a sample from a subject at a first time point;
b) obtaining a second sample from the same subject at a second time point;
c) determining the level of expression of one or more genes for each of the time points, wherein the one or more genes is CDC42, CORO1A, AURKB, CBX5, IQGAP1, TERF2, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1;
d) predicting the likelihood of survival of the subject by comparing the expression level of one or more of the genes at the first time point to the expression level of one or more of the genes at the second time point, wherein a change in the expression level of one or more of the genes is predictive of survival.

2. The method of claim 1, wherein an increase in the expression of CDC42 or TERF2 indicates a decreased likelihood of survival.

3. The method of claim 1, wherein an increase in the expression of CORO1A, AURKB, CBX5, IQGAP1, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1 indicates an increased likelihood of survival.

4. The method according to claim 1, wherein the subject is human.

5. The method of claim 1, further comprising:
a) determining telomere length of the subject; and
b) correlating the telomere length with survival with telomere length in an age matched population of the subject.

6. The method according to claim 5, wherein telomere length is the average telomere length.

7. A method for predicting the likelihood of survival of a subject comprising determining the presence of one or more single nucleotide polymorphisms (SNPs) with an LOD score of greater than 3.5 with modulated expression of the IQGAP1 gene, wherein the presence of one or more single nucleotide polymorphisms (SNPs) with an LOD score of greater than 3.5 with modulated expression of the IQGAP1 gene is predictive of survival.

8. The method of claim 7, wherein the one or more single nucleotide polymorphisms is rs716175, rs937793, rs3862432, rs3930162, rs17263706, rs3862434, rs8033595, rs12915189, rs7498042, rs12901137, rs12910489, rs12914286, rs7403002, rs11857476, rs7403440, rs10438448, rs4344687 or rs716175.

9. The method according to claim 1, wherein the subject is human.

10. The method of claim 7, further comprising
a) obtaining a sample from a subject at a first time point;
b) obtaining a second sample from the same subject at a second time point;
c) determining the level of expression of one or more genes for each of the time points, wherein the one or more genes is CDC42, CORO1A, AURKB, CBX5, IQGAP1, TERF2, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1;
d) comparing the expression level of one or more of the genes at the first time point to the expression level of one or more of the genes at the second time point, wherein a change in the expression level of one or more of the genes is predictive of survival.

11. The method of claim 10, wherein an increase in the expression of CDC42 or TERF2 indicates a decreased likelihood of survival.

12. The method of claim 10, wherein an increase in the expression of CORO1A, AURKB, CBX5, IQGAP1, CDKN3, PBK, PBXK, AIVD1, RNF13, CDC42EP3, F8, LRRFIP1, SHOC2, GEN, RNPEP, PDIA5, HEXB, ZDHHC13, RAD51AP1, GGH, CETN3, GFPT1, or PRIVI1 indicates an increased likelihood of survival.

13. The method of claim 7, further comprising:
a) determining telomere length of the subject; and
b) correlating the telomere length with survival with telomere length in an age matched population of the subject.

14. The method according to claim 13, wherein telomere length is the average telomere length.

15. The method according to claim 14, wherein the average telomere length is determined by polymerase chain reaction.

16. The method according to claim 13, wherein the telomere length is determined from blood.

17. The method according to claim 13, wherein the telomere length is determined from lymphoid cells.

18. The method according to claim 17, wherein the lymphoid cells comprise T cells.

19. The method according to claim 13, wherein the age matched population is within about 10 years of the age.

20. The method according to claim 13, wherein the age matched population is within about 5 years of the age.


SEQUENCE LISTING (51 sequences)

SEQ ID NO: 1 - DNA, 52 bp, Artificial Sequence
  Description of Artificial Sequence note = synthetic construct
agtcagaccaaagtatggggaaagagnggtaaacgaggcagggaacaccaga

SEQ ID NO: 2 - DNA, 52 bp, Artificial Sequence
  Description of Artificial Sequence note = synthetic construct
tgaggggtggagaatggatctcaacgnacaaaaaggagatatccagcacaac

SEQ ID NO: 3 - DNA, 52 bp, Artificial Sequence
  Description of Artificial Sequence note = synthetic construct
tacatgcactgtatatgtctgtatacntatggcggatccaatctttcaccca

SEQ ID NO: 4 - DNA, 52 bp, Artificial Sequence
  Description of Artificial Sequence note = synthetic construct
ccctgacaggtgtggccatgagtaatntacagactctcttttaaaagcaaaa

SEQ ID NO: 5 - DNA, 52 bp, Artificial Sequence
  Description of Artificial Sequence note = synthetic construct
ttggctgtgaagagcaacttgtgttgnggaaccagaggactaactcagcata

SEQ ID NO: 6 - DNA, 52 bp, Artificial Sequence
  Description of Artificial Sequence note = synthetic construct
agtctcaaagttctttttctagctcantgagtctaaatttcaaaagaggaaa

SEQ ID NO: 7 - DNA, 52 bp, Artificial Sequence
  Description of Artificial Sequence note = synthetic construct
accaaatgtgagccagctgcggagcantgcgtcagagtttcaggtacctcca

SEQ ID NO: 8 - DNA, 52 bp, Artificial Sequence
  Description of Artificial Sequence note = synthetic construct
acacaatgcttgatgctggagatacantgacccagacaggcacagtcactgc

SEQ ID NO: 9 - DNA, 52 bp, Artificial Sequence
  Description of Artificial Sequence note = synthetic construct
tcactttcatgctgcttttcggtgatntttctaggctattcttgcatgttta

SEQ ID NO: 10 - DNA, 52 bp, Artificial Sequence
  Description of Artificial Sequence note = synthetic construct
cacccacagctaaactgtggttgggantgactgagtgattgtagttctgaaa

SEQ ID NO: 11 - DNA, 52 bp, Artificial Sequence
  Description of Artificial Sequence note = synthetic construct
cccaagtagttgggactgtaggagtgngacatcatgccaagctaatttaaat

SEQ ID NO: 12 - DNA, 52 bp, Artificial Sequence
  Description of Artificial Sequence note = synthetic construct
cttgaatgggaccacaggcctgtgcanaagaagggagggtgaactgatatct

SEQ ID NO: 13 - DNA, 52 bp, Artificial Sequence
  Description of Artificial Sequence note = synthetic construct
gttgcccactgtcttcctcacctaccngggctttccagagtattttttgcag

SEQ ID NO: 14 - DNA, 52 bp, Artificial Sequence
  Description of Artificial Sequence note = synthetic construct
tttaatgaatgaatcaatgagtgattnacaaacacattagttatatatgtgg

SEQ ID NO: 15 - DNA, 52 bp, Artificial Sequence
  Description of Artificial Sequence note = synthetic construct
tataaattgatacattaaatacacacngagagacatatatttagagggtatt

SEQ ID NO: 16 - DNA, 52 bp, Artificial Sequence
  Description of Artificial Sequence note = synthetic construct
gggtaaagggagaatctagcaaggganagatagatgtcgagctctagacaaa

SEQ ID NO: 17 - DNA, 52 bp, Artificial Sequence
  Description of Artificial Sequence note = synthetic construct
cttatttttactgcctcatgaaagtangacttacgtaccataaaatccactc

SEQ ID NO: 18 - DNA, 53 bp, Artificial Sequence
  Description of Artificial Sequence note = synthetic construct
agtcagaccaaagtatggggaaagagctggtaaacgaggcagggaacaccaga

SEQ ID NO: 19 - DNA, 52 bp, Artificial Sequence
  Description of Artificial Sequence note = synthetic construct
agtcagaccaaagtatggggaaagagtggtaaacgaggcagggaacaccaga

SEQ ID NO: 20 - DNA, 52 bp, Artificial Sequence
  Description of Artificial Sequence note = synthetic construct
tgaggggtggagaatggatctcaacgaacaaaaaggagatatccagcacaac

SEQ ID NO: 21 - DNA, 52 bp, Artificial Sequence
  Description of Artificial Sequence note = synthetic construct
tgaggggtggagaatggatctcaacggacaaaaaggagatatccagcacaac

SEQ ID NO: 22 - DNA, 52 bp, Artificial Sequence
  Description of Artificial Sequence note = synthetic construct
tacatgcactgtatatgtctgtatacctatggcggatccaatctttcaccca

SEQ ID NO: 23 - DNA, 52 bp, Artificial Sequence
  Description of Artificial Sequence note = synthetic construct
tacatgcactgtatatgtctgtatacttatggcggatccaatctttcaccca

SEQ ID NO: 24 - DNA, 52 bp, Artificial Sequence
  Description of Artificial Sequence note = synthetic construct
ccctgacaggtgtggccatgagtaatatacagactctcttttaaaagcaaaa

SEQ ID NO: 25 - DNA, 52 bp, Artificial Sequence
  Description of Artificial Sequence note = synthetic construct
ccctgacaggtgtggccatgagtaatgtacagactctcttttaaaagcaaaa

SEQ ID NO: 26 - DNA, 52 bp, Artificial Sequence
  Description of Artificial Sequence note = synthetic construct
ttggctgtgaagagcaacttgtgttgaggaaccagaggactaactcagcata

SEQ ID NO: 27 - DNA, 52 bp, Artificial Sequence
  Description of Artificial Sequence note = synthetic construct
ttggctgtgaagagcaacttgtgttgcggaaccagaggactaactcagcata

SEQ ID NO: 28 - DNA, 52 bp, Artificial Sequence
  Description of Artificial Sequence note = synthetic construct
agtctcaaagttctttttctagctcaatgagtctaaatttcaaaagaggaaa

SEQ ID NO: 29 - DNA, 52 bp, Artificial Sequence
  Description of Artificial Sequence note = synthetic construct
agtctcaaagttctttttctagctcagtgagtctaaatttcaaaagaggaaa

SEQ ID NO: 30 - DNA, 52 bp, Artificial Sequence
  Description of Artificial Sequence note = synthetic construct
accaaatgtgagccagctgcggagcaatgcgtcagagtttcaggtacctcca

SEQ ID NO: 31 - DNA, 52 bp, Artificial Sequence
  Description of Artificial Sequence note = synthetic construct
accaaatgtgagccagctgcggagcagtgcgtcagagtttcaggtacctcca

SEQ ID NO: 32 - DNA, 52 bp, Artificial Sequence
  Description of Artificial Sequence note = synthetic construct
acacaatgcttgatgctggagatacaatgacccagacaggcacagtcactgc

SEQ ID NO: 33 - DNA, 52 bp, Artificial Sequence
  Description of Artificial Sequence note = synthetic construct
acacaatgcttgatgctggagatacagtgacccagacaggcacagtcactgc

SEQ ID NO: 34 - DNA, 52 bp, Artificial Sequence
  Description of Artificial Sequence note = synthetic construct
tcactttcatgctgcttttcggtgatgtttctaggctattcttgcatgttta

SEQ ID NO: 35 - DNA, 52 bp, Artificial Sequence
  Description of Artificial Sequence note = synthetic construct
tcactttcatgctgcttttcggtgatttttctaggctattcttgcatgttta

SEQ ID NO: 36 - DNA, 52 bp, Artificial Sequence
  Description of Artificial Sequence note = synthetic construct
cacccacagctaaactgtggttgggactgactgagtgattgtagttctgaaa

SEQ ID NO: 37 - DNA, 52 bp, Artificial Sequence
  Description of Artificial Sequence note = synthetic construct
cacccacagctaaactgtggttgggattgactgagtgattgtagttctgaaa

SEQ ID NO: 38 - DNA, 52 bp, Artificial Sequence
  Description of Artificial Sequence note = synthetic construct
cccaagtagttgggactgtaggagtgcgacatcatgccaagctaatttaaat

SEQ ID NO: 39 - DNA, 52 bp, Artificial Sequence
  Description of Artificial Sequence note = synthetic construct
cccaagtagttgggactgtaggagtgtgacatcatgccaagctaatttaaat

SEQ ID NO: 40 - DNA, 52 bp, Artificial Sequence
  Description of Artificial Sequence note = synthetic construct
cttgaatgggaccacaggcctgtgcagaagaagggagggtgaactgatatct

SEQ ID NO: 41 - DNA, 52 bp, Artificial Sequence
  Description of Artificial Sequence note = synthetic construct
cttgaatgggaccacaggcctgtgcataagaagggagggtgaactgatatct

SEQ ID NO: 42 - DNA, 52 bp, Artificial Sequence
  Description of Artificial Sequence note = synthetic construct
gttgcccactgtcttcctcacctacccgggctttccagagtattttttgcag

SEQ ID NO: 43 - DNA, 52 bp, Artificial Sequence
  Description of Artificial Sequence note = synthetic construct
gttgcccactgtcttcctcacctacctgggctttccagagtattttttgcag

SEQ ID NO: 44 - DNA, 52 bp, Artificial Sequence
  Description of Artificial Sequence note = synthetic construct
tttaatgaatgaatcaatgagtgattcacaaacacattagttatatatgtgg

SEQ ID NO: 45 - DNA, 52 bp, Artificial Sequence
  Description of Artificial Sequence note = synthetic construct
tttaatgaatgaatcaatgagtgattgacaaacacattagttatatatgtgg

SEQ ID NO: 46 - DNA, 52 bp, Artificial Sequence
  Description of Artificial Sequence note = synthetic construct
tataaattgatacattaaatacacacagagagacatatatttagagggtatt

SEQ ID NO: 47 - DNA, 52 bp, Artificial Sequence
  Description of Artificial Sequence note = synthetic construct
tataaattgatacattaaatacacacggagagacatatatttagagggtatt

SEQ ID NO: 48 - DNA, 52 bp, Artificial Sequence
  Description of Artificial Sequence note = synthetic construct
gggtaaagggagaatctagcaagggacagatagatgtcgagctctagacaaa

SEQ ID NO: 49 - DNA, 52 bp, Artificial Sequence
  Description of Artificial Sequence note = synthetic construct
gggtaaagggagaatctagcaagggatagatagatgtcgagctctagacaaa

SEQ ID NO: 50 - DNA, 52 bp, Artificial Sequence
  Description of Artificial Sequence note = synthetic construct
cttatttttactgcctcatgaaagtacgacttacgtaccataaaatccactc

SEQ ID NO: 51 - DNA, 52 bp, Artificial Sequence
  Description of Artificial Sequence note = synthetic construct
cttatttttactgcctcatgaaagtatgacttacgtaccataaaatccactc